(19) United States
(12) Patent Application Publication
(10) Pub. No.: US 2024/0408203 A1
(43) Pub. Date: Dec. 12, 2024

(54) ENGINEERING NK CELLS WITH A CAR CONSTRUCT WITH OPTIMAL SIGNALING

(71) Applicant: BOARD OF REGENTS, THE UNIVERSITY OF TEXAS SYSTEM, Austin, TX (US)

(72) Inventors: Katy REZVANI, Houston, TX (US); May DAHER, Houston, TX (US); Rafet BASAR, Houston, TX (US); Sunil ACHARYA, Houston, TX (US); Nadima UPRETY, Houston, TX (US); Ana Karen NUNEZ CORTES, Houston, TX (US); Emily ENSLEY, Houston, TX (US)

(73) Assignee: BOARD OF REGENTS, THE UNIVERSITY OF TEXAS SYSTEM, Austin, TX (US)

(21) Appl. No.: 18/699,702

(22) PCT Filed: Oct. 19, 2022
(86) PCT No.: PCT/US2022/078331

§ 371 Date: Apr. 9, 2024

Related U.S. Application Data
(60) Provisional application No. 63/257,608, filed on Oct. 20, 2021.

(51) Int. Cl. A61K 39/00; A61P 35/00; C07K 14/705; C07K 14/725; C07K 16/28; C12N 5/0783

(52) U.S. Cl. CPC A61K 39/4613; A61K 39/4631; A61K 39/464429; A61P 35/00; C07K 14/7051; C07K 14/70521; C07K 16/2896; C12N 5/0646; C07K 2317/53; C07K 2319/02; C07K 2319/03; C12N 2510/00

(57) ABSTRACT

Embodiments of the disclosure encompass particular chimeric antigen receptor constructs that comprise optionally a hinge, one of the CD28 transmembrane domain or the DAP10 transmembrane domain, DAP10 costimulatory domain, and CD3zeta. In particular embodiments, the chimeric antigen receptor is expressed by natural killer (NK) cells, and in some cases the NK cells are further modified, such as to express one or more cytokines and optionally a suicide gene.

This application claims priority to U.S. Provisional Patent Application Ser. No. 63/257,608, filed Oct. 20, 2021.

The instant application contains a Sequence Listing which has been submitted in XML format and is hereby incorporated by reference in its entirety. Said XML copy, created on Oct. 11, 2022, is named MDAC_1299WO_Sequence_Listing_ST26.xml and is 13,926 bytes in size.

### I. TECHNICAL FIELD

Embodiments of the disclosure include at least the fields of cell biology, molecular biology, immunology, and medicine, including cancer medicine.

### II. BACKGROUND

In recent years, adoptive cellular therapy using autologous T cells transduced with chimeric antigen receptor (CAR) has proven to be a very powerful approach for the treatment of cancer, leading to FDA approvals in B cell leukemia/lymphoma.¹⁻³ However, challenges remain, including uncoupling cytotoxicity against tumor cells from systemic toxicity, finding solutions for target antigen negative relapses, and developing universal off-the-shelf cell therapy products to avoid the logistic hurdles of generating autologous products while managing issues that arise with allogeneic T cell products.⁴ Natural killer (NK) cells are attractive contenders for CAR engineering because they mediate effective cytotoxicity against tumor cells and, unlike T-cells, lack the potential to cause graft-versus-host disease (GVHD) in the allogeneic setting.⁵ Thus, NK cells could be made available as an off-the-shelf cellular therapy product for immediate clinical use. CAR-NK cells also retain their intrinsic capacity to recognize and target tumor cells through their native receptors, thus in principle, making disease escape through downregulation of the CAR target antigen less likely than is observed with CAR-T cells.⁶ Cord blood (CB) is a readily available “off-the-shelf” source of allogeneic NK cells that (as one example) can be expanded to large, highly functional doses using GMP-compliant universal antigen presenting cells (uAPC) that are K562 cells engineered to express CD48, 4-1BBL, and membrane-bound IL-21 (mbIL21).⁷

The present disclosure satisfies a longfelt need in the art of adoptive cell therapy to provide highly effective NK cells that are able to target desired antigens through particular CAR configurations.

### BRIEF SUMMARY

Embodiments of the disclosure include methods and compositions related to adoptive cell therapies for individuals in need thereof, including cell therapies where the cells are modified NK cells. The modified NK cells express particular synthetic proteins that render them particularly effective for treatment of a particular medical condition, such as by allowing the NK cells to have enhanced efficacy against cells to which the synthetic proteins are targeted.

In particular embodiments, the present disclosure concerns chimeric antigen receptor constructs that comprise the DAP10 co-stimulatory domain (which is more relevant to NK cell biology, such as compared to T cell biology) in combination with either a DAP10 transmembrane domain or CD28 transmembrane domain, and/or optionally in combination with a CD28 hinge. Such DAP10-comprising CAR constructs result in enhanced CAR-NK cell anti-tumor activity compared to other co-stimulatory domains, such as CD28 costimulatory domains that are more specific to T cell biology. In specific embodiments, such constructs are utilized to improve adoptive CAR-NK cellular therapies, and by enhancing the potency this enables use of a lower number of CAR-NK (or CAR-T cells, in alternative cases) to individuals in need thereof to reduce the risk of toxicity.

Particular embodiments of the disclosure encompass adoptive cellular therapy with CAR-NK cells (or other alternative CAR vehicles) to treat patients with any type of hematologic malignancy, solid cancer, and/or infectious disease.

Embodiments of the disclosure include polynucleotides that encode a fusion protein, said fusion protein comprising: (a) optionally a CD28 hinge; and (b1) a CD28 transmembrane domain, or (b2) a DAP10 transmembrane domain; and (c) a DAP10 costimulatory domain. In specific embodiments, the fusion protein is further defined as a chimeric antigen receptor (CAR). In some cases, the CAR further comprises one or more antigen binding domains, including wherein an antigen binding domain targets a cancer antigen (solid tumors or hematological malignancies) or an infectious agent. In specific cases, the CAR further comprises CD3zeta, such as one that comprises SEQ ID NO:3. The polynucleotide may encode the CD28 transmembrane domain comprised in SEQ ID NO:1. In certain embodiments, the CAR further comprises one or more additional costimulatory domains, such as one or more additional costimulatory domains selected from the group consisting of CD28, DAP12, 4-1BB, NKG2D, 2B4, and a combination thereof. The CAR may or may not further comprise a signal peptide, such as a signal peptide from CD8, CD27, granulocyte-macrophage colony-stimulating factor receptor (GMSCF-R), Ig heavy chain (IgH), CD3, or CD4, as examples.

In particular embodiments, polynucleotides of the disclosure include those that further encode one or more additional polypeptides of interest. The sequence encoding one or more additional polypeptides of interest and the sequence encoding the CAR may be separated on the polynucleotide by a 2A element or IRES. In certain cases, the additional polypeptide of interest is one or more therapeutic proteins and/or proteins that enhances cell activity, expansion, cytotoxicity, and/or persistence. In some cases, the additional polypeptide of interest is a suicide gene product, one or more cytokines (IL-15, IL-2, IL-12, IL-18, IL-21, IL-23, and/or IL-7, for example), and/or one or more human or viral proteins that enhance proliferation, expansion and/or metabolic fitness. In cases wherein a cytokine is IL-15, the IL-15 sequence may comprise SEQ ID NO:8.

In particular embodiments, a vector of any kind comprises any polynucleotide of the disclosure, including a viral vector, such as an adenoviral vector, adeno-associated viral vector, lentiviral vector, or retroviral vector, or a non-viral vector, such as a plasmid. Also encompassed in the disclosure are cells of any kind that comprise any polynucleotide encompassed herein and/or any vector encompassed herein. The cell may be a stem cell or an immune cell, or mixture thereof. Specific immune cells include the following: natural killer (NK) cell, T cell, gamma delta T cell, alpha beta T cell, invariant NKT (iNKT) cell, B cell, macrophage, mesenchymal stromal cell, dendritic cell, or a mixture thereof. When the immune cell is a NK cell, the NK cell may be derived from cord blood, peripheral blood, induced pluripotent stem cells, hematopoietic stem cells, bone marrow, or from a cell line, such as an NK cell derived from the NK-92 cell line. The NK cell may be derived from a cord blood mononuclear cell. The NK cell may be a CD56+NK cell. In specific embodiments, the NK cell expresses a recombinant cytokine, such as IL-15, IL-2, IL-12, IL-18, IL-21, IL-7, and/or IL-23. Also included herein are populations of immune cells or stem cells that express one or more CAR molecules of the disclosure. When more than one type of CAR molecule is expressed by the cells, the CARs may target different antigens, such as to enhance the ability to specifically bind the intended cell(s). The population may or may not comprise a mixture of cells of any kind.

Embodiments of the disclosure include methods of killing cancer cells in an individual, comprising administering to the individual an effective amount of any cells harboring any polynucleotide encompassed herein and/or any cells harboring any vector encompassed herein. In specific embodiments, the cells harboring the polynucleotide are immune cells, such as NK cells, T cells, gamma delta T cells, alpha beta T cells, iNKT cells, B cells, macrophages, dendritic cells, or a mixture thereof. The immune cells may comprise NK cells, wherein the NK cells are derived from cord blood (including CB mononuclear cells), peripheral blood, induced pluripotent stem cells, hematopoietic stem cells, bone marrow, from a cell line, or a mixture thereof. The immune cells may be autologous or allogeneic with respect to the individual. In particular embodiments of the methods, the cells harboring the polynucleotide and/or cells harboring the vector are administered to the individual once or more than once, and the duration of time between administrations of the cells harboring the polynucleotide to the individual may be 1-24 hours, 1-7 days, 1-4 weeks, 1-12 months, or one or more years. Methods may further comprise the step of providing to the individual an effective amount of an additional therapy, such as surgery, radiation, gene therapy, immunotherapy, or hormone therapy. The cells harboring the polynucleotide and/or the cells harboring the vector may be administered to the individual by infusion, injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1B. Phenotyping using mass cytometry panel. 1A. TSNE phonograph plots showing different clusters in non-transduced NT (left) and CD5CAR-NK CD28TMDAP10CD3z transduced NK cells. The clusters are numbered with a color code legend as indicated below the phenographs. The 2 new clusters (#8 and #11) expressed in CARCD5 NK cells are highlighted by the circles and the rectangles. 1B. Heatmap showing the normalized expression of various markers (indicated on the X axis) in each cluster (indicated on the Y axis). The activation, cytotoxicity and maturation markers with high expression in clusters #8 and #11 are highlighter with the rectangles.

FIGS. 2A-2C. Isoplexis single cell secretome data showing polyfunctionality of CD5 CAR-NK cells with DAP10 costimulatory domain. 2A. Bar graph showing percent polyfunctionality of different CD5 CAR-NK cells compared to non-transduced (NT) NK cells. 2B. Bar graph showing the polyfunctionality strength index among the different CD5 CAR-NK cells compared to non-transduced (NT) NK cells. 2C. Polyfunctionality heatmap showing which constructs have the highest ability to secrete various permutations of cytokines at the single cell level.

FIGS. 3A-3C. Incucyte killing assay experiment with multiple tumor rechallenges. 3A. Schematic diagram showing one embodiment of an experimental design and methodology of the Incucyte killing assay rechallenge experiment. 3B. Graph showing the red count (y-axis; a measure of live tumor count) after each tumor rechallenge (indicated by the arrow) among the various CD5 CAR-NK cell conditions. 3C. Graph showing the percent confluence (a measure of tumor abundance) after each tumor rechallenge (indicated by the arrows).

FIGS. 4A-4B. Seahorse metabolic assay measuring oxygen consumption rate (OCR) and extracellular acidification rate (ECAR) among various CD5 CAR-NK cells. 4A. Graph showing OCR among the various CD5 CAR-NK cell designs compared to non-transduced (NT) NK cells. 4B. Graph showing ECAR among the various CD5 CAR-NK cell designs compared to non-transduced (NT) NK cells.

FIGS. 5A-5C. CD5 CAR-NK cells with DAP10 costimulatory domain show good activity in a PDX mouse model of mantle cell lymphoma. 5A. Bar graph showing the absolute number of CD45+CD5+ cells (in the subcutaneous tumor in mice who received tumor alone (left) vs tumor plus CD5CAR-NK (right) in the subcutaneous tumor. 5B. Bar graph showing the absolute number of CD45+CD5+ cells in the subcutaneous tumor in mice who received tumor alone (left) vs tumor plus CD5CAR-NK (right) in the spleen. 5C. Bar graph showing the absolute number of CD45+CD5+ cells in the subcutaneous tumor in mice who received tumor alone (left) vs tumor plus CD5CAR-NK (right) in the bone marrow.

FIGS. 6A-6B. CD27 CAR-NK cells with DAP10 costimulatory domain improve tumor control and survival in an NSG mouse model of acute myeloid leukemia (THP-1 transduced with firefly luciferase (FFLuc). 6A. Series of bioluiminescent imaging (BLI) showing tumor burden as luminescence of THP-1 FFLuc among the various groups of mice. 6B. Survival curve showing the survival of the various groups of mice over time.

FIGS. 7A-7B. FIG. 7A illustrates various construct identifications and corresponding transduction efficiency (FIG. 7B). CB-NK cells were transduced with various CD5 CAR constructs, as shown in FIG. 7A, and the transduction efficiency was measured by flow cytometry. The transduction efficiency is based on percent positive cells (FIG. 7B). In FIG. 7B, the bars from left to right in the bar graph correspond to those in the legend as read from top to bottom.

FIG. 8 provides one example of an experimental plan for mice injection with various CD5 constructs and a corresponding timeline. The schematic shows testing of the in vivo antitumor activity of various CD5 CAR NK cells against the T lymphoblastoid cell line CCRF-CEM as a target.

FIGS. 9A-9B. FIGS. 9A and 9B show that mice treated with anti-CD5 CAR NK with IgG1 hinge cells survive significantly longer than NT NK cell and Tumor alone. Bioluminescence images are shown of mice in each group (FIG. 9A), and quantification of luciferase signal is shown in FIG. 9B.

FIGS. 10A-10B. FIGS. 10A and 10B demonstrate that mice treated with anti-CD5 CAR NK with CD28 hinge reduce tumor burden significantly compared to Tumor alone, NT NK cells and CD5 CAR NK cells with IgG1 hinge. Bioluminescence images of mice in each group is provided in FIG. 10A, and quantification of luciferase signal is shown in FIG. 10B.

FIG. 11. CD5 CAR-NK cells with DAP10 signaling show evidence of high proliferative and metabolic advantage at the single cell transcriptomic level. Heatmap showing pathway enrichment analysis of scRNAseq data comparing CD5CAR-DAP10-CD3z, CD5CAR-CD3z and NT NK cells. N=2.

FIG. 12. CD5 CAR-NK cells with DAP10 signaling display enrichment of TFs related to API complex and BATF family at the single cell epigenetic level. Volcano plot showing TF enrichment analysis of scATACseq data comparing CD5CAR-DAP10-CD3z and CD5CAR-CD3z. N=2.

FIGS. 13A-13B. CD5 CAR-NK cells with DAP10 signaling show enhanced activation at the proteomic level by RPPA. 13A. Heatmap of RPPA analysis showing Log2 protein expression data of CD5CAR-DAP10-CD3z and CD5CAR-CD3z normalized to NT NK cells before stimulation (unstim) and following stimulation with CD5 target antigen for 2 min and 15 min. 13B. Pathway network analysis showing interaction between various protein pathways relating to proliferation, stemness, metabolic activity, immune synapse formation and memory features. N=2.

FIGS. 14A-14B. CD5 CAR-NK cells with DAP10 signaling persist and have the ability to mount a recall response following tumor rechallenge in vivo. 14A. Schematic diagram showing the details of the experimental plan of the in vivo mouse model showing timing of irradiation, timing of CD5+CCRF tumor injection, timing of CD5 CAR-NK cells infusion and timing of rechallenge with CD5+ CCRF tumor (transduced with FFLuc-GFP). 14B. FACS plots showing pre-rechallenge (left sided panels) and post-rechallenge flow cytometry data (right sided panels) showing human CD45+ gate followed by NK cell gate (CD56+ and GFP−). This shows that CD5 CAR-NK cells expand after tumor re-challenge and can mount a recall response against CD5+CCRF tumor.

### DETAILED DESCRIPTION EXAMPLES OF DEFINITIONS

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/of” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

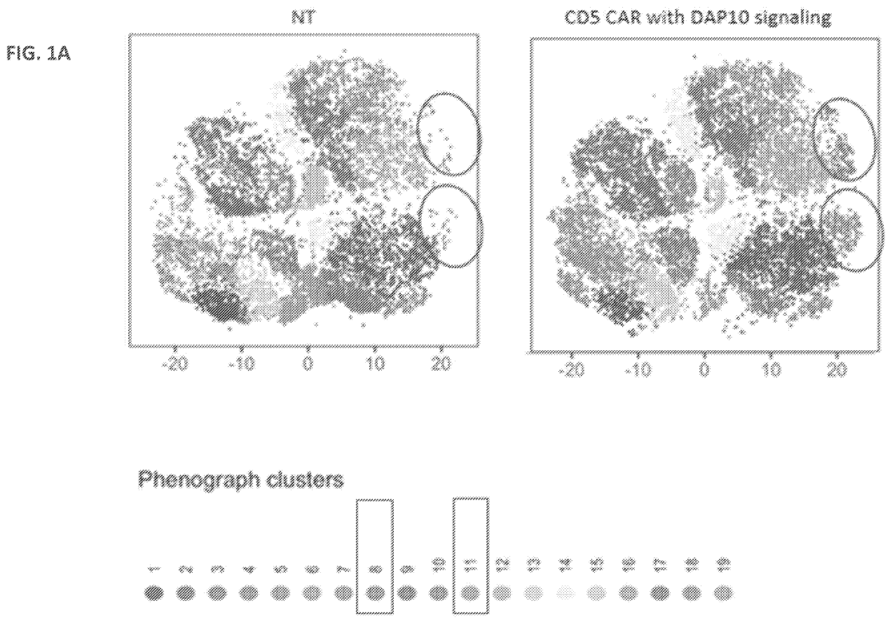

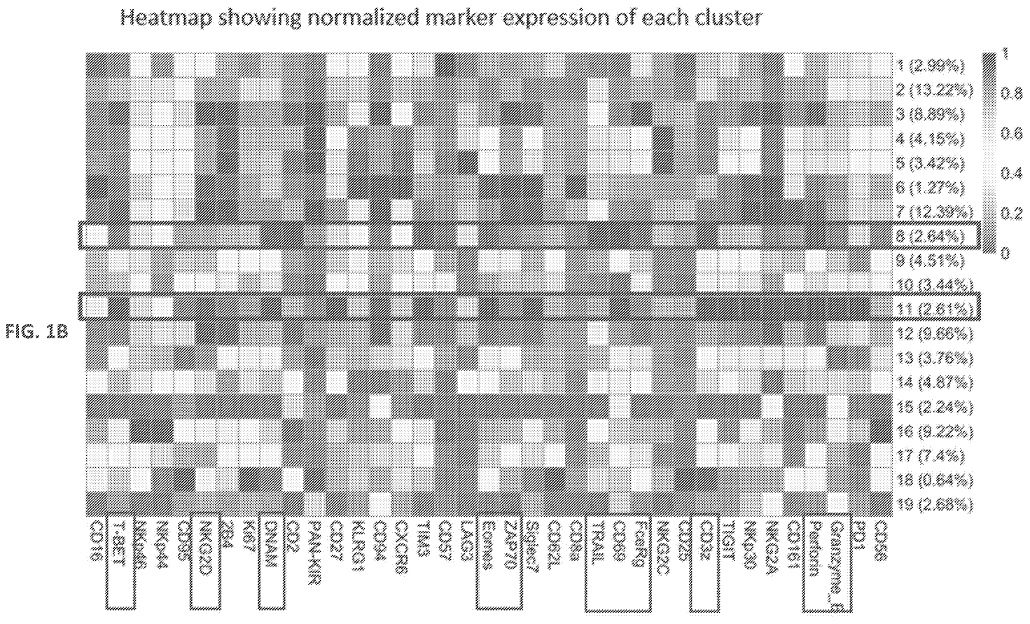

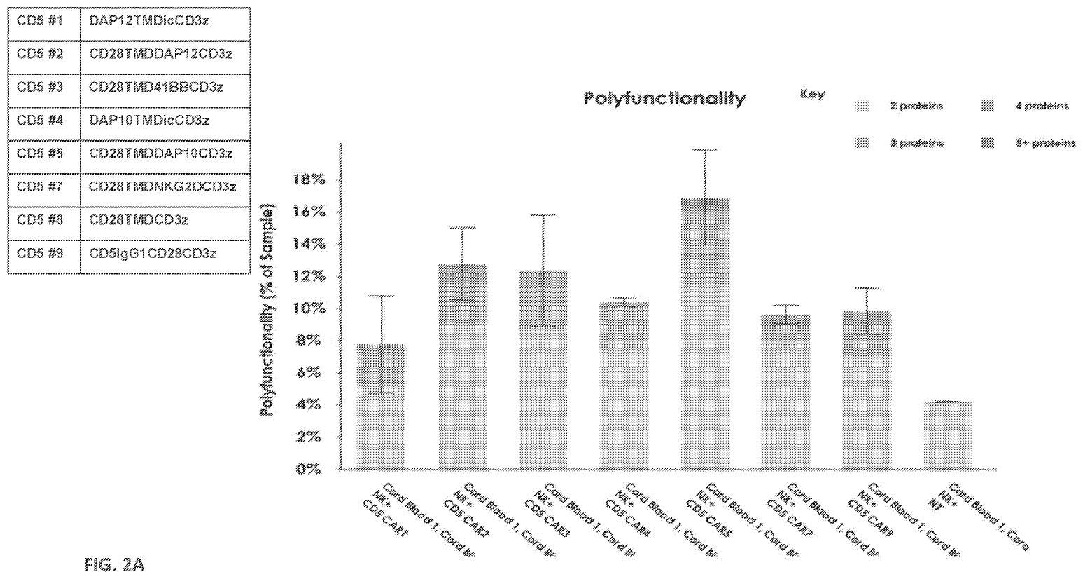

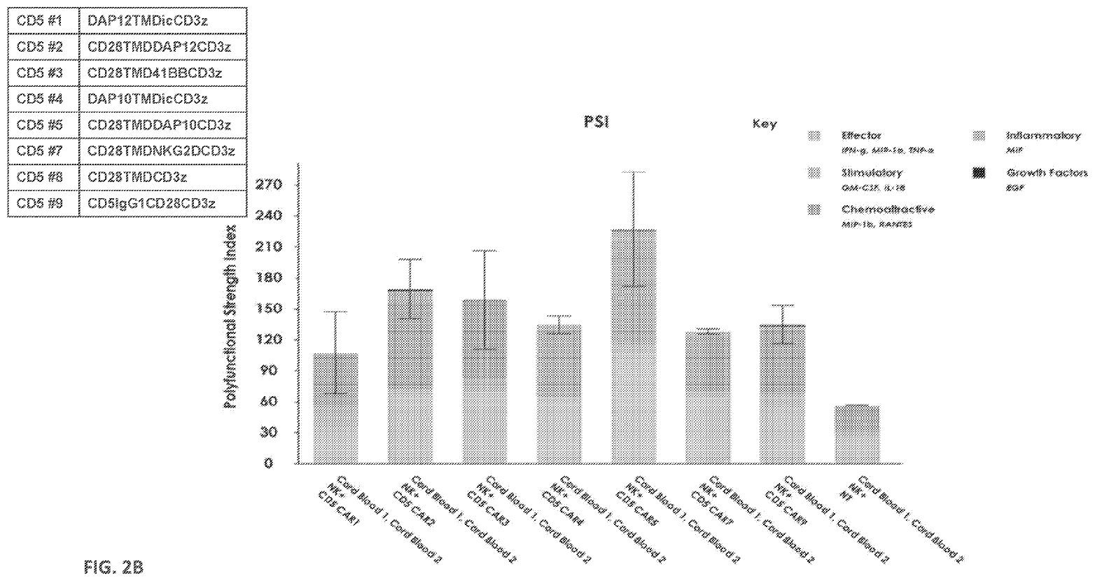

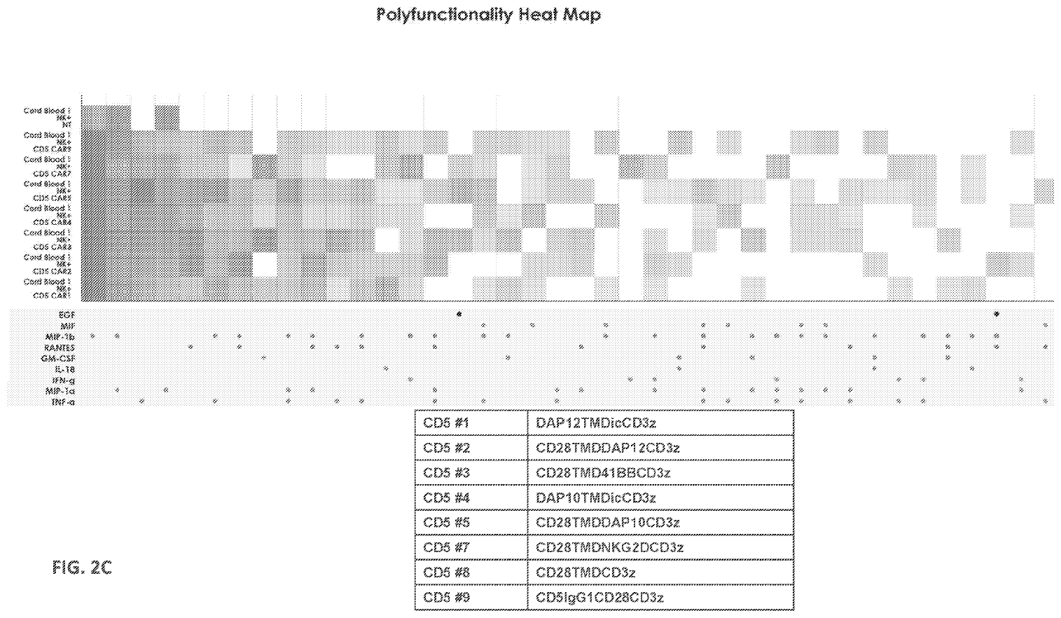

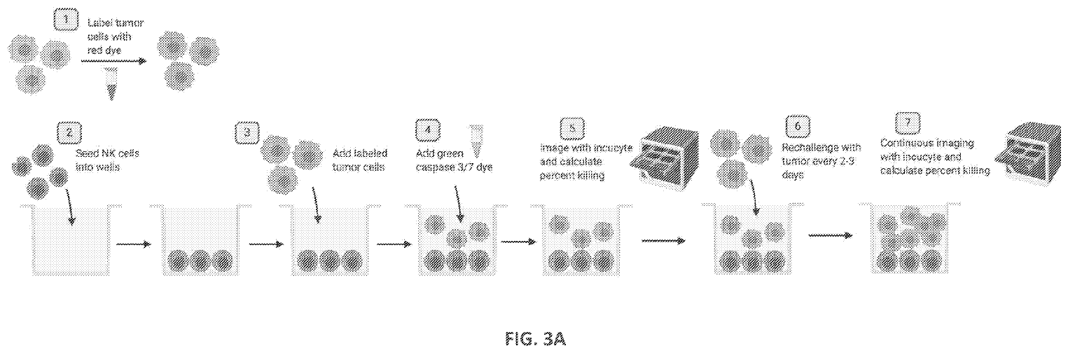

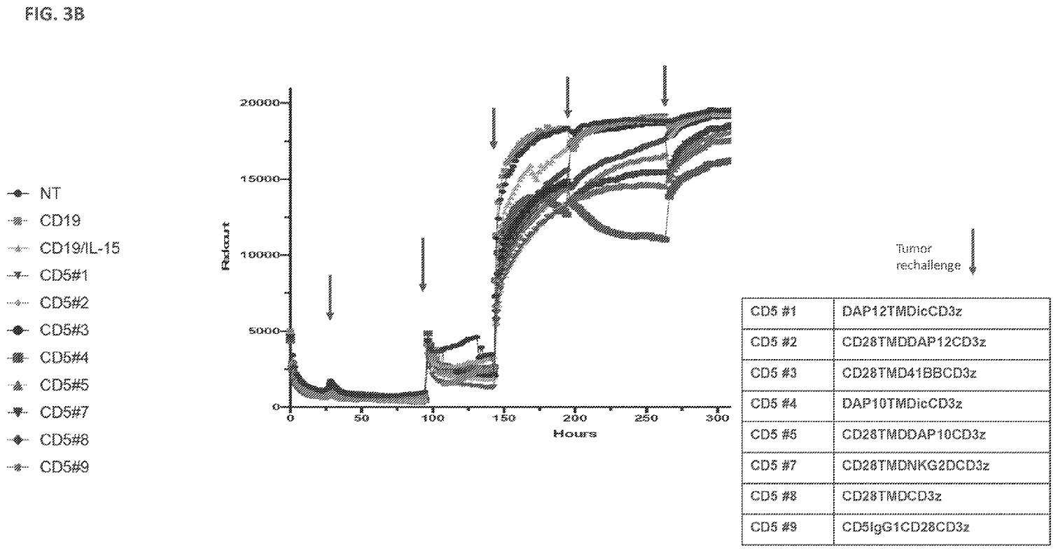

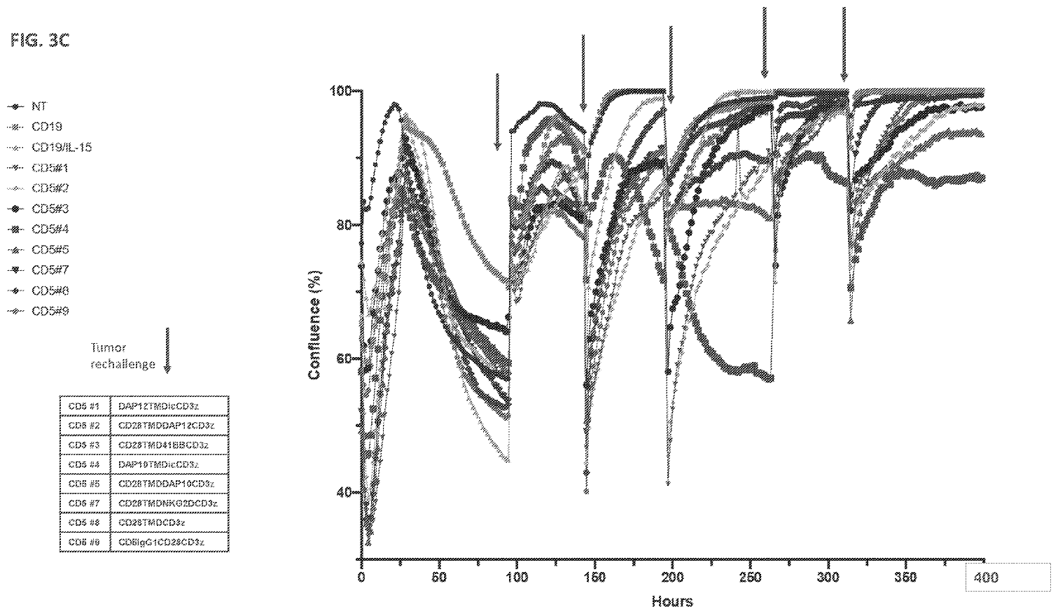

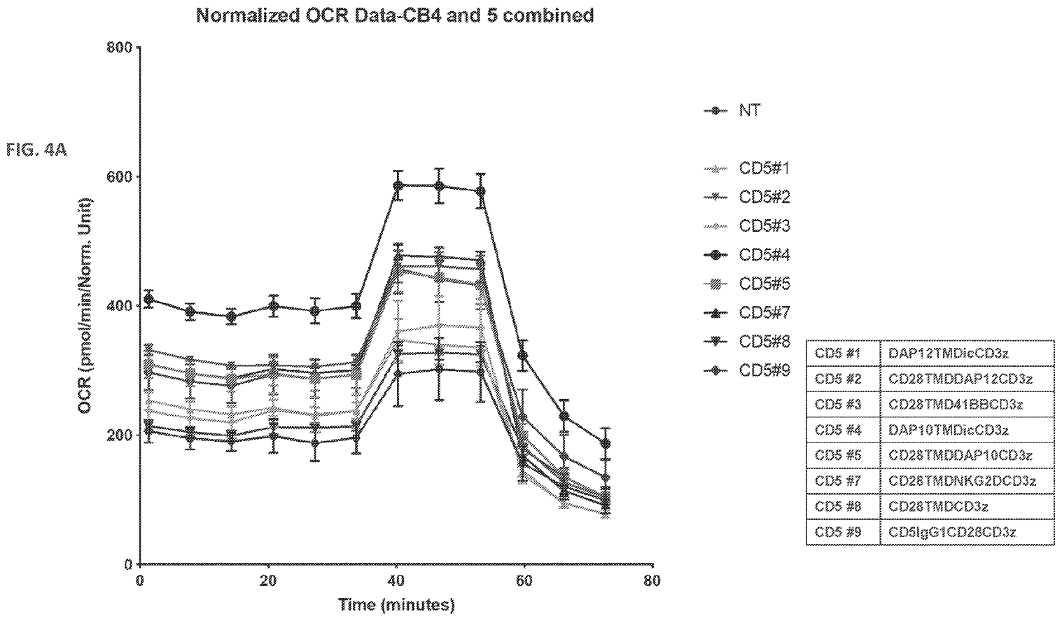

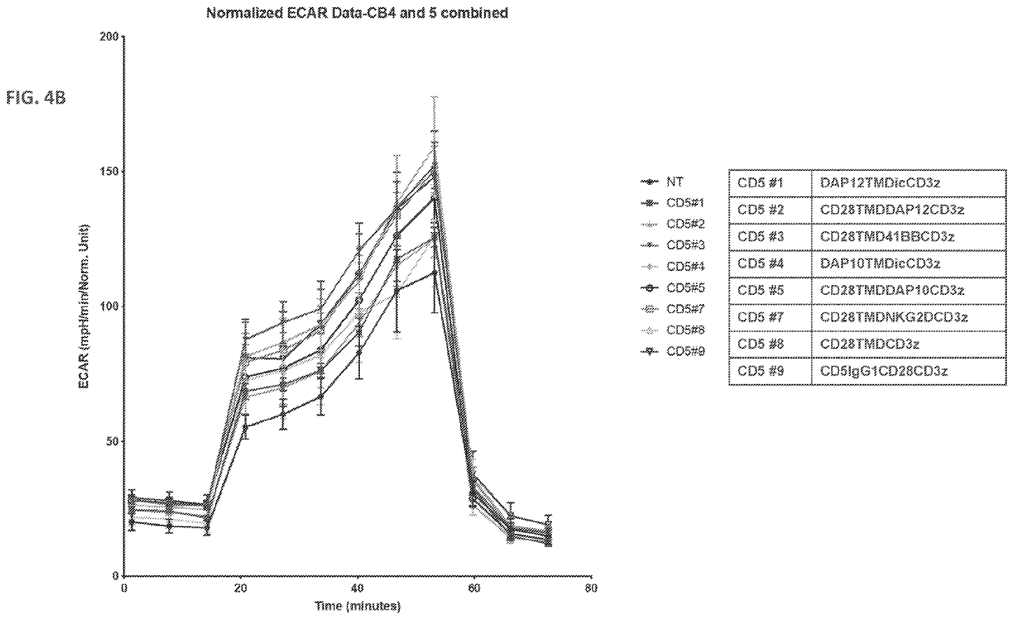

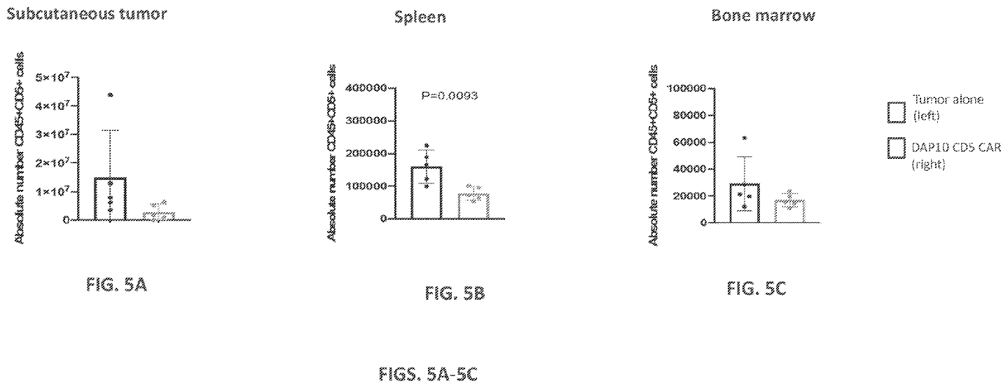

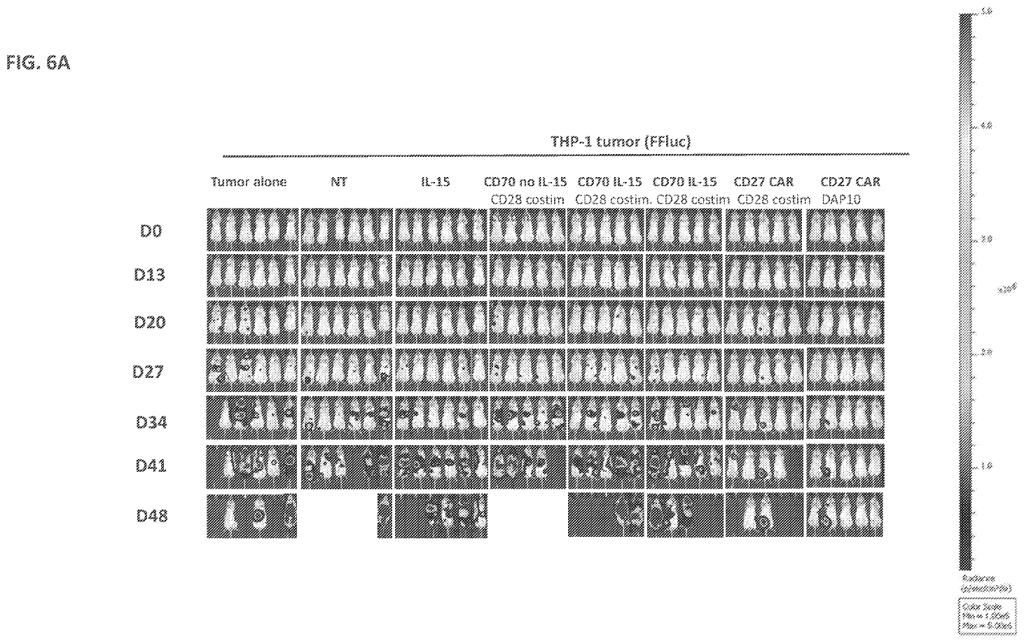

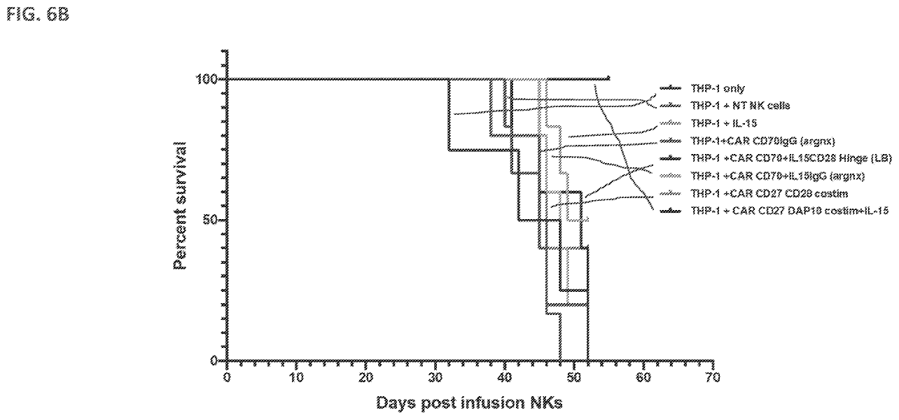

The present disclosure concerns methods and compositions in which chimeric antigen receptor constructs at least in some cases are better suited to use in NK cells because they have one or more components that are more relevant to NK cells, as opposed to biology that would be suited to other immune cells, including T cells. In one example, given that CD28 is a co-stimulatory molecule relevant to T cell biology and not present in NK cells, the inventors developed other CAR vectors with alternative co-stimulatory molecules more relevant to NK cell biology, such as DAP10, DAP12, NKG2D and/or 4-1BB, as examples. As encompassed herein, different CAR designs with the various co-stimulatory molecules were characterized with either CD5 scFv or with CD27 extracellular domain as the antigen binding domain, although these are merely examples. DAP10 co-stimulatory domain confers a more activated phenotype to the CAR-NK cells, with specific clusters identified by mass cytometry showing increased expression of activation markers, such as DNAM, NKG2D, CD3z and ZAP70; cytotoxicity markers such as TRAIL, Granzyme B and Perforin; and maturation markers such as Eomes and T-bet (FIG. 1). Moreover, CAR-NK cells with DAP10 co-stimulatory domain and either a DAP10 or CD28 transmembrane domain showed a higher polyfunctionality compared to CAR-NK cells with other co-stimulatory domains (FIG. 2). Importantly, CAR-NK cells directed against CD5 with DAP10 co-stimulatory domain had the ability to kill CCRF T-ALL cell line after multiple tumor rechallenges, whereas CAR-NK cells with other co-stimulatory molecules lost the ability to kill CCRF with later tumor rechallenges, likely due to functional exhaustion (FIG. 3). From a metabolism perspective, CAR-NK cells with DAP10 co-stimulatory domain showed a higher metabolic fitness with higher oxidative phosphorylation, as evidenced by a higher oxygen consumption rate (OCR) and a higher glycolytic capacity and as evidenced by a higher extracellular acidification rate (ECAR) (FIG. 4). This can correlate with their enhanced potency, in particular embodiments. This translated to an enhanced anti-tumor activity of the DAP10 construct in a PDX mouse model of mantle cell lymphoma testing the efficacy of CD5 CAR-NK cells (FIG. 5) and in an NSG mouse model of acute myeloid leukemia (THP-1) testing the efficacy of CD70 CAR-NK cells (FIG. 6). As shown herein, DAP10, which is an important adaptor molecule downstream of the NK activating receptor NKG2D, can serve as a potent co-stimulatory domain for CAR-NK cells and can enhance their metabolic fitness and their anti-tumor activity in vitro and in vivo.

### I. Genetically Engineered Receptors

The present disclosure concerns genetically engineered receptors that utilize particular component(s) for enhanced efficacy over other genetically engineered receptors that lack the particular components. In particular embodiments, the receptors optionally comprise a hinge. In cases wherein the receptor comprises an scFv as at least part of an extracellular domain, the receptor may comprise a hinge between the scFv and the transmembrane domain. In specific examples, the receptor may or may not comprise a hinge when the extracellular domain of the receptor lacks an scFv, such as comprises at least part of an extracellular domain of an endogenous or other receptor. In specific cases, the receptors comprise at least a CD28 hinge. In some cases, the receptors comprise at least a CD28 transmembrane domain. In certain cases, the receptors comprise at least a DAP10 transmembrane domain. In specific cases, the receptors comprise at least a DAP10 costimulatory domain.

In specific embodiments, the enhanced receptors comprise (including in the form of a fusion protein) the following:
(a) optionally a hinge; and
(b1) a CD28 transmembrane domain, or
(b2) a DAP10 transmembrane domain;
(c) a DAP10 costimulatory domain; and
(d) CD3zeta.

Thus, in specific cases, the receptor comprises a CD28 hinge (or CD8alpha hinge or IgG1 hinge, as examples), a CD28 transmembrane domain, and a DAP10 costimulatory domain. In other cases, the receptor comprises a CD28 hinge, a DAP10 transmembrane domain, and a DAP10 costimulatory domain. In specific embodiments the components of (a) (optionally), (b1) or (b2), (c) and (d) are in the form of a fusion protein and in an N-terminal to C-terminal direction are in the order of (a) (optionally), (b1) or (b2), (c), and (d). In specific cases the fusion protein lacks (a).

In specific embodiments, the components of (a) (optionally), (b1) or (b2), (c), and (d) in the form of a fusion protein further comprise one or more antigen binding domains, including as a CAR configuration. In specific embodiments one or more antigen binding domains in an N-terminal to C-terminal direction are on the N-terminal side of (a) (optionally), (b1) or (b2), (c), and (d) (in that order). In specific embodiments, the fusion protein as a genetically engineered receptor consists essentially of, or consists of, one or more antigen binding domains, (a) (optionally), (b1) or (b2), (c), and (d). That is, in specific cases the genetically engineered receptor lacks any other costimulatory domains than DAP10, although in alternative cases the genetically engineered receptor comprises one or more costimulatory domains other than DAP10. In such alternative cases wherein the genetically engineered receptor comprises one or more other costimulatory domains than DAP10, the other costimulatory domain(s) may or may not be a costimulatory domain that is present in a protein that is naturally found in NK cells and not T cells. Specific examples include CD28, DAP12, 4-1BB, NKG2D, 2B4, a combination thereof, and so forth.

The components of (a) (optionally), (b1) or (b2), (c), and (d) may be expressed on a single polynucleotide as a fusion protein and in particular embodiments the fusion protein comprises one or more antigen binding domains. The polynucleotide may be isolated or may be comprised in a vector of any kind, including viral or non-viral. In specific embodiments, the vectors are present in any type of cell including immune cells, such as NK cells, T cells, gamma delta T cells, alpha beta T cells, iNKT cells, B cells, macrophages, dendritic cells, or a mixture thereof. Suitable methods of modification of cells are known in the art. See, for instance, Sambrook and Ausubel, supra. For example, the cells may be transduced to express the genetically engineered receptor having antigenic specificity for a cancer antigen or an antigen of an infectious agent using transduction techniques described in Heemskerk et al., 2008 and Johnson et al., 2009.

In some embodiments, the cells comprise one or more nucleic acids introduced via genetic engineering that encode one or more genetically engineered receptors) and express the genetically engineered products of such nucleic acids. In particular embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric). They may be the product of the hand of man.

Exemplary antigen receptors, including CARs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

In particular embodiments, the genetically engineered receptor comprises one or more antigen binding domains and the components of (a), (b1) or (b2), and (c). In specific embodiments, the genetically engineered receptor is a CAR, and in some embodiments the antigen binding domain is an antibody or functional fragment thereof. In other cases, the antigen binding domain of the CAR is not an antibody or functional fragment thereof but instead is a natural ligand for a receptor. The CAR may be a single polypeptide that is bispecific by comprising two or more antigen binding domains, one of which that binds a desired antigen and the other of which binds another, non-identical antigen.

In some embodiments, the engineered antigen receptors include CARs, including activating or stimulatory CARs, or costimulatory CARs (see WO2014/055668. The CARs generally include an extracellular antigen (or ligand) binding domain linked to one or more intracellular signaling components, in some aspects via the components of (a), (b1) or (b2), and (c). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a specific CAR polypeptide comprising the components of (a) (optionally), (b1) or (b2), (c), and (d), including in some cases a CAR that has been humanized to reduce immunogenicity (hCAR). In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the disclosure includes a full-length antigen-specific CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is a antigen-specific scFv encoded by a sequence that is optimized for human codon usage for expression in human cells.

In some embodiments, an antigen-specific CAR is constructed with specificity for the antigen, such as the antigen being expressed on a diseased cell type (a cancer cell or cell infected with an infectious agent). Thus, the CAR typically includes in its extracellular portion one or more antigen-binding molecules, such as one or more antigen-binding fragments, domains, antibody variable domains, ligands, receptors, and/or antibody molecules of any kind. One of skill in the art is able to generate antibodies, including scFvs against the antigen based on knowledge at least of the polypeptide and routine practices, although numerous anti-antigen scFvs and monoclonal antibodies may already be present in the art. In some embodiments, the antigen-specific scFv is an scFV from one or more of antibody clones.

In some embodiments, the antigen-specific CAR includes an antigen-binding portion or portions of an antibody molecule, such as a single-chain antibody fragment (scFv) derived from the variable heavy (VH) and variable light (VL) chains of a monoclonal antibody (mAb). In specific embodiments, the antibody or functional fragment thereof is or is derived from a known antibody. The antibody may also be one that is generated de novo against the antigen, and the scFv sequence may be obtained, or derived, from such de novo antibodies.

In certain embodiments, the CAR comprises an extracellular domain that is or comprises a natural ligand or natural receptor for the target antigen or receptor. In some embodiments, the CAR comprises an extracellular domain that is or comprises a VH and/or VL from an antibody targeting the antigen.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding domain is linked to the CD28 or DAP10 transmembrane domain and in specific cases is also linked to the DAP10 costimulatory domain. In some instances, the CD28 or DAP10 transmembrane domain is modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic CD28 or DAP10 transmembrane domain.

In some embodiments, the CAR nucleic acid comprises a sequence encoding other than DAP10 and, optionally CD3zeta. In addition to a primary T cell activation signal, such as may be initiated by CD3ζ and/or FcεRIγ, besides DAP10 an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, NKG2D, 2B4, CD2, CD28, CD27, 4-1BB, OX40, ICOS, (CD278), CD30, HVEM, CD40, LFA-1 (CD11a/CD18), and/or ICAM-1, although in specific alternative embodiments any one of these listed may be excluded from use in the CAR.

### II. Examples of Specific CAR Embodiments

In particular embodiments, specific CAR molecules are encompassed herein comprising (a) optionally, a hinge; and (b1) a CD28 transmembrane domain, or (b2) a DAP10 transmembrane domain; (c) a DAP10 costimulatory domain; and (d) CD3zeta. In some cases, the CAR further comprises an antigen binding domain of any kind and that may be a scFv of any kind. In cases wherein an scFv is utilized in the extracellular domain of the CAR, the variable heavy chain and the variable light chain for the particular scFv may be in any order in N-terminal to C-terminal direction. For example, the variable heavy chain may be on the N-terminal side of the variable light chain, or vice versa. The scFv that binds the antigen in the CAR may or may not be codon optimized. In particular embodiments, a vector encodes an antigen-specific CAR comprising (a) (optionally), (b1) or (b2), (c), and (d) and also encodes one or more other molecules. For example, a vector may encode such a CAR and also may encode another protein of interest, such as one or more other engineered antigen receptors, a suicide gene, and/or one or more particular cytokines.

On the same molecule, the CAR may comprise one or more antigen-specific extracellular domains, such as for targeting two different antigens, and there may be a linker between the two antigen-specific extracellular domains.

In particular embodiments of specific CAR molecules, a CAR utilizes DAP10 but also utilizes CD28, DAP12, 4-1BB, NKG2D, or other costimulatory domains, including those encompassed herein (which may be referred to herein as an intracytoplasmic domain).

Examples of specific sequence embodiments for the CAR are provided below, in no particular order:

CD28 transmembrane domain amino acid sequence:

(SEQ ID NO: 1)
FWVLVVVGGVLACYSLLVTVAFIIFWV

Any polypeptide encompassed by the present disclosure may comprise SEQ ID NO:1, or a sequence that is at least 70, 75, 80, 85, 90, 95, 96, 97, 98, 99, or more % identical to SEQ ID NO:1.

One or more intracellular domains (which may also be referred to herein as signal activation domains or costimulatory domains, in appropriate cases) may or may not be utilized in specific CARs of the disclosure. Specific examples include intracellular domains from DAP10 and, in particular cases, CD3 zeta.

Examples of particular intracellular domains that may be used in a CAR of the disclosure are as follows:

An example of a DAP10 intracellular domain amino acid sequence:

(SEQ ID NO: 2)
LCARPRRSPAQEDGKVYINMPGRG

Any polypeptide encompassed by the present disclosure may comprise SEQ ID NO:2, or a sequence that is at least 70, 75, 80, 85, 90, 95, 96, 97, 98, 99, or more % identical to SEQ ID NO:2.

An example of a CD3zeta intracellular domain amino acid sequence:

(SEQ ID NO: 3)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTAT
KDTYDALHMQALPPRG

Any polypeptide encompassed by the present disclosure may comprise SEQ ID NO:3, or a sequence that is at least 70, 75, 80, 85, 90, 95, 96, 97, 98, 99, or more % identical to SEQ ID NO:3.

In some cases, the CAR further comprises an intracellular domain other than DAP10 (and in some cases CD3zeta), such as follows:

4-1BB intracellular domain amino acid sequence:

(SEQ ID NO: 4)
KRGRKKLLYIFKQPFMRPVQTTQEEDGCSCRFPEEEEGGCEL

Any polypeptide encompassed by the present disclosure may comprise SEQ ID NO:4, or a sequence that is at least 70, 75, 80, 85, 90, 95, 96, 97, 98, 99, or more % identical to SEQ ID NO:4.

DAP12 intracellular domain amino acid sequence:

(SEQ ID NO: 5)
YFLGRLVPRGRGAAEAATRKQRITETESPYQELQGQRSDVYSDLNTQRP
YYK

Any polypeptide encompassed by the present disclosure may comprise SEQ ID NO:5, or a sequence that is at least 70, 75, 80, 85, 90, 95, 96, 97, 98, 99, or more % identical to SEQ ID NO:5.

NKG2D intracellular domain amino acid sequence:

(SEQ ID NO: 6)
SANERCKSKVVPCRQKQWRTSFDSKKLDLNYNHFESMEWSHRSRRGRIW
GM

CD28 intracellular domain amino acid sequence:

(SEQ ID NO: 14)
RSKRSRLLHSDYMNMTPRRPGPTRKHYQPYAPPRDFAAYRS

Any polypeptide encompassed by the present disclosure may comprise SEQ ID NO:6, or a sequence that is at least 70, 75, 80, 85, 90, 95, 96, 97, 98, 99, or more % identical to SEQ ID NO:6.

In some embodiments of the CARs, there is a hinge region between the one or more extracellular antigen binding domains and the transmembrane domain, and in specific cases this occurs when an scFv is used in the CAR but not when an scFv is lacking in the CAR. In specific embodiments, the hinge is of a particular length, such as 10-20, 10-15, 11-20, 11-15, 12-20, 12-15, or 15-20 amino acids in length, for example. In specific embodiments, the hinge is a CD28 hinge. In specific cases, one can modify the identity or length of the CD28 hinge to enhance efficiency of the CAR. See, for example, Hudecek et al. (2014) and Jonnalagadda et al. (2015). In specific embodiments, the hinge is from CD28, CD8alpha or IgG1.

An example of a CD28 Hinge amino acid sequence includes:

(SEQ ID NO: 7)
IEVMYPPPYLDNEKSNGTIIHVKGKHLCPSPLFPGPSKP

Any polypeptide encompassed by the present disclosure may comprise SEQ ID NO:7 or a sequence that is at least 70, 75, 80, 85, 90, 95, 96, 97, 98, 99, or more % identical to SEQ ID NO:7.

In specific embodiments, the following examples of expression constructs for the CAR may be utilized. Although these CARs target CD5, other antigens may be targeted.

CD5 #1 comprises IgG hinge with the DAP12 transmembrane domain, DAP12 costimulatory domain, and CD3 zeta.

CD5 #2 comprises IgG hinge with the CD28 transmembrane domain, DAP12 costimulatory domain, and CD3 zeta.

CD5 #3 comprises IgG hinge with the CD28 transmembrane domain, 4-1BB costimulatory domain, and CD3zeta

CD5 #4 comprises IgG hinge with the DAP10 transmembrane domain, DAP10 costimulatory domain, and CD3 zeta

CD5 #5 comprises IgG hinge with the CD28 transmembrane domain, DAP10 costimulatory domain, and CD3 zeta.

CD5 #7 comprises IgG hinge with the CD28 transmembrane domain, NKG2D costimulatory domain, and CD3zeta.

CD5 #8 comprises IgG hinge with the CD28 transmembrane domain and CD3zeta.

CD5 #9 comprises the IgG1 hinge, CD28 transmembrane domain, CD28 costimulatory domain, and CD3zeta.

CD5 #10 comprises the CD28 hinge, CD28 transmembrane domain, DAP10 costimulatory domain, and CD3zeta.

CD5 #11 comprises the CD28 hinge, DAP10 transmembrane domain, DAP10 costimulatory domain, and CD3zeta.

CD5 #12 comprises the CD28 hinge, CD28 transmembrane domain, DAP12 costimulatory domain, and CD3zeta.

CD5 #13 comprises the CD28 hinge, CD28 transmembrane domain, CD28 costimulatory domain, and CD3zeta.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015).

In particular aspects, the antigens are associated with cancer and include CD19, EBNA, CD123, HER2, CA-125, TRAIL/DR4, CD20, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD70, TROP-2, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a infectious agent, including a pathogenic microorganism or from an opportunistic pathogenic microorganism (also called herein an infectious disease microorganism), such as a virus, fungus, parasite, protozoan, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gpl20, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (HI, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of vims replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al, 2006), envelope glycoprotein B (gB), gH, gN, and ppl50. As would be understood by the skilled person, CMV proteins for use as antigens described herein may be identified in public databases such as GENBANK®, SWISS-PROT®, and TREMBL® (see e.g., Bennekov et al, 2004; Loewendorf et al, 2010; Marschall et al, 2009).

Antigens derived from Epstein-Ban vims (EBV) that are contemplated for use in certain embodiments include EBV lytic proteins gp350 and gpl 1O, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al, 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al, 1999).

Antigens derived from an influenza virus that are contemplated for use in certain embodiments include hemagglutinin (HA), neuraminidase (NA), nucleoprotein (NP), matrix proteins M1 and M2, NS 1, NS2 (NEP), PA, PB 1, PB 1-F2, and PB2.

Exemplary viral antigens also include, but are not limited to, adenovirus polypeptides, alphavirus polypeptides, calicivims polypeptides (e.g., a calicivims capsid antigen), coronavirus polypeptides, distemper virus polypeptides, Ebola virus polypeptides, enterovirus polypeptides, flavivirus polypeptides, hepatitis vims (AE) polypeptides (a hepatitis B core or surface antigen, a hepatitis C vims E1 or E2 glycoproteins, core, or non-structural proteins), herpesvirus polypeptides (including a herpes simplex virus or varicella zoster virus glycoprotein), infectious peritonitis vims polypeptides, leukemia vims polypeptides, Marburg vims polypeptides, orthomyxovirus polypeptides, papilloma vims polypeptides, parainfluenza vims polypeptides (e.g., the hemagglutinin and neuraminidase polypeptides), paramyxovirus polypeptides, parvovirus polypeptides, pestivims polypeptides, picorna vims polypeptides (e.g., a poliovims capsid polypeptide), pox vims polypeptides (e.g., a vaccinia vims polypeptide), rabies vims polypeptides (e.g., a rabies vims glycoprotein G), reovims polypeptides, retrovirus polypeptides, and rotavirus polypeptides.

In certain embodiments, the antigen may be a bacterial antigen. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al, 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al, 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®.

Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis FI and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Tricho strongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSAl c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### III. Optional Proteins

In some embodiments, one or more other proteins are utilized with the particular CAR of the disclosure. The one or more other proteins may be utilized for any reason, including to facilitate efficacy of the CAR itself and/or to facilitate efficacy of any kind of cells expressing the CAR. In some cases, the other protein facilitates treatment of an individual receiving cells expressing the CAR as therapy, whether or not the other protein(s) directly or indirectly impact activity of the CAR or the cells. In some cases, the other protein is a suicide gene, one or more cytokines, or both. In specific embodiments, one or more other proteins are produced from a vector and ultimately are produced as two separate polypeptides. For example, the CAR and the other protein(s) may be separated by a 2A sequence or by an IRES, for example.

In specific embodiments, one or more cytokines, such as IL-15, are utilized in conjunction with the CAR.

IL-15 amino acid sequence:

(SEQ ID NO: 8)
ISKPHLRSISIQCYLCLLLNSHFLTEAGIHVFILGCFSAGLPKTEANWV
NVISDLKKIEDLIQSMHIDATLYTESDVHPSCKVTAMKCFLLELQVISL
ESGDASIHDTVENLIILANNSLSSNGNVTESGCKECEELEEKNIKEFLQ
SFVHIVQMFINTS

Any polypeptide encompassed by the present disclosure may comprise SEQ ID NO:8 or a sequence that is at least 70, 75, 80, 85, 90, 95, 96, 97, 98, 99, or more % identical to SEQ ID NO:8.

In specific embodiments, a suicide gene product such as caspase 9 (e.g., inducible caspase 9) is utilized in conjunction with the CAR.

Example caspase 9 amino acid sequence:

(SEQ ID NO: 9)
MLEGVQVETISPGDGRTFPKRGQTCVVHYTGMLEDGKKVDSSRDRNKPF
KFMLGKQEVIRGWEEGVAQMSVGQRAKLTISPDYAYGATGHPGIIPPHA
TLVFDVELLKLESGGGSGVDGFGDVGALESLRGNADLAYILSMEPCGHC
LIINNVNFCRESGLRTRTGSNIDCEKLRRRFSSLHFMVEVKGDLTAKKM
VLALLELAQQDHGALDCCVVVILSHGCQASHLQFPGAVYGTDGCPVSVE
KIVNIFNGTSCPSLGGKPKLFFIQACGGEQKDHGFEVASTSPEDESPGS
NPEPDATPFQEGLRTFDQLDAISSLPTPSDIFVSYSTFPGFVSWRDPKS
GSWYVETLDDIFEQWAHSEDLQSLLLRVANAVSVKGIYKQMPGCFNFLR
KKLFFKTSAS

Any polypeptide encompassed by the present disclosure may comprise SEQ ID NO:9 or a sequence that is at least 70, 75, 80, 85, 90, 95, 96, 97, 98, 99, or more % identical to SEQ ID NO:9.

In cases where the CAR and another protein in the same vector are intended to be produced into two different polypeptides, a specific 2A sequence may be utilized.

E2A amino acid sequence may be utilized as follows:

(SEQ ID NO: 10)
QCTNYALLKLAGDVESNPGP

Other 2A examples may be utilized and are as follows:

T2A:
(SEQ ID NO: 11)
EGRGSLLTCGDVEENPGP
P2A:
(SEQ ID NO: 12)
ATNFSLLKQAGDVEENPGP
F2A:
(SEQ ID NO: 13)
VKQTLNFDLLKLAGDVESNPGP

The disclosure also encompasses specific CAR molecules, including for expression in any type of immune effector cells.

In a vector, the CAR may be expressed with IL-15, such as may be separated from the CAR by a 2A sequence.

### A. Cytokines

One or more cytokines may be utilized with one or more of the disclosed genetically engineered receptors, such as CARs comprising (a) optionally a hinge; and (b1) a CD28 transmembrane domain, or (b2) a DAP10 transmembrane domain; and (c) a DAP10 costimulatory domain. In some cases, one or more cytokines are present on the same vector molecule as the engineered receptor, although in other cases they are on separate vector molecules. In particular embodiments, one or more cytokines are co-expressed from the same vector as the engineered receptor. One or more cytokines may be produced as a separate polypeptide from the antigen-specific receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the one or more cytokines are IL-15, IL-12, IL-2, IL-18, IL-21, IL-23, IL-7, or combination thereof. NK cells expressing IL-15 may be utilized and are capable of continued supportive cytokine signaling, which is useful for their survival post-infusion.

In specific embodiments, NK cells express one or more exogenously provided cytokines. The cytokine may be exogenously provided to the NK cells because it is expressed from an expression vector within the cell and/or because it is provided in a culture medium of the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene. The cytokine may be expressed as a separate polypeptide molecule from a suicide gene and as a separate polypeptide from an engineered receptor of the cell. In some embodiments, the present disclosure concerns co-utilization of CAR and/or TCR vectors with IL-15, particularly in NK cells.

### B. Suicide Genes

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The antigen-targeting cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product. A “suicide gene product” describes a protein or polypeptide encoded by a suicide gene.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP). In some embodiments, an inducible caspase 9 (iC9) is used. An example iC9 is described in, for example, Yagyu S, et al. Mol Ther. 2015 September; 23(9):1475-85, incorporated by reference herein in its entirety.

In particular embodiments, vectors that encode the CAR, or any vector in a NK cell encompassed herein, include one or more suicide genes. The suicide gene may or may not be on the same vector as a CAR. In cases wherein the suicide gene is present on the same vector as the CAR, the suicide gene and the CAR may be separated by an IRES or 2A element, for example.

### C. Other Receptors

In some embodiments, the cells that comprise the CAR may express one or more other receptors, including other CAR molecules that may or may not comprise any one or more components encompassed herein, one or more cytokine receptors, one or more chemokine receptors (e.g., as modifications to enhance trafficking and homing to tumors sites such as CXCR1 and CXCR2 to enhance trafficking to CXCL8-producing tumors), and/or one or more synthetic TCRs. In cases wherein the other receptor targets an antigen, such as a cancer antigen, the other receptor may or may not target the same antigen as the CARs of the disclosure.

### IV. Vectors

The CARs comprising (a) a CD28 hinge; and (b1) a CD28 transmembrane domain, or (b2) a DAP10 transmembrane domain; and (c) a DAP10 costimulatory domain may be delivered to the recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the genetically engineered receptor and also requires transduction of another gene or genes into the cell, such as a suicide gene and/or cytokine and/or an optional therapeutic gene product, the antigen-targeting receptor, suicide gene, cytokine, and optional therapeutic gene may or may not be comprised on or with the same vector. In some cases, the CAR, suicide gene, cytokine, and optional therapeutic gene are expressed from the same vector molecule, such as the same viral vector molecule. In such cases, the expression of the CAR, suicide gene, cytokine, and optional therapeutic gene may or may not be regulated by the same regulatory element(s). When the CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, a vector encoding the genetically engineered receptor comprising (a) CD28 hinge, (b1) CD28 transmembrane domain or (b2) DAP10 transmembrane domain, and (c) DAP10 costimulatory domain, also comprises sequence that encodes an optional suicide gene, optional cytokine, and/or optional therapeutic gene, including expressed from a multicistronic vector (The term “cistron” as used herein refers to a nucleic acid sequence from which a gene product may be produced). In specific embodiments, the multicistronic vector encodes the receptor, the suicide gene, and at least one cytokine, and/or engineered receptor, such as a T-cell receptor and/or an additional CAR. In some cases, the multicistronic vector encodes at least one CAR, at least one non-secretable TNF-alpha mutant, and at least one cytokine. The cytokine may be of a particular type of cytokine, such as human or mouse or any species. In specific cases, the cytokine is IL15, IL12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (IL-15, IL-7, IL-21, IL-23, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprise a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CARs comprising (a) CD28 hinge, (b1) CD28 transmembrane domain or (b2) DAP10 transmembrane domain, and (c) DAP10 costimulatory domain and optionally associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence—5′ LTR—psi packaging sequence—genetic cargo of interest—3′ LTR—pUC19 sequence. This system may also be applied to other viral and non-viral vectors, including but not limited lentivirus, adenovirus AAV, as well as non-viral plasmids.

### V. Cells

The present disclosure encompasses immune cells or stem cells of any kind that harbor at least one vector that encodes the genetically engineered receptor comprising (a) CD28 hinge, (b1) CD28 transmembrane domain or (b2) DAP10 transmembrane domain, and (c) DAP10 costimulatory domain and that also may encode at least one cytokine and/or at least one suicide gene. In some cases, different vectors encode the CAR vs. encodes the suicide gene and/or cytokine. The immune cells, including NK cells, may be derived from cord blood (including pooled cord blood from multiple sources), peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56+NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the CAR is expanded ex vivo. The clone selected for expansion demonstrates the capacity to specifically recognize and lyse the antigen-expressing target cells. The recombinant immune cells may be expanded by stimulation with IL-2, or other cytokines that bind the common gamma-chain (e.g., IL-7, IL-12, IL-15, IL-18, IL-21, IL-23, and others). The recombinant immune cells may be expanded by stimulation with artificial antigen presenting cells. In a further aspect, the genetically modified cells may be cryopreserved.

Embodiments of the disclosure encompass cells that express one or more CARs and one or more suicide genes as encompassed herein. The NK cell comprises a recombinant nucleic acid that encodes one or more CARs and one or more engineered nonsecretable, membrane bound TNF-alpha mutant polypeptides, in specific embodiments. In specific embodiments, in addition to expressing one or more CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the CAR, optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more receptors and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more CARs and/or one or more suicide genes and/or one or more cytokines. The one or more CARs and/or one or more suicide genes and/or one or more cytokines may be separate polypeptides.

The immune cells may be produced with the one or more CARs and/or one or more suicide genes and/or one or more cytokines for the intent of being modular with respect to a specific purpose. For example, cells may be generated, including for commercial distribution, expressing a CAR and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating antigen-positive cells, including antigen-positive cancer or infectious agent-infected cells, may obtain or generate suicide gene-expressing cells (or heterologous cytokine-expressing cells) and modify them to express a receptor comprising an antigen-specific scFv, or vice versa.

In particular embodiments, NK cells are utilized, and the genome of the transduced NK cells expressing the one or more CARs and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

### VI. Methods of Treatment

In various embodiments, diseased or other cells expressing a desired on their surface are targeted for the purpose of improving a medical condition in an individual that has the medical condition or for the purpose of reducing the risk or delaying the severity and/or onset of the medical condition in an individual. In specific cases, cancer cells expressing the endogenous antigen are targeted for the purpose of killing the cancer cells. In other cases, cells infected with an infectious agent are targeted for the purpose of killing the infected cells.

In particular embodiments, CAR constructs, nucleic acid sequences, vectors, immune cells and so forth as contemplated herein, and/or pharmaceutical compositions comprising the same, are used for the prevention, treatment or amelioration of a disease, such as a cancerous disease. In particular embodiments, the pharmaceutical composition of the present disclosure may be particularly useful in preventing, ameliorating and/or treating cancer, including cancers that express a particular antigen and that may or may not be solid tumors, for example.

The immune cells for which the receptor is utilized may be NK cells, T cells, gamma delta T cells, alpha beta T cells, or NKT or invariant NKT (iNKT), or invariant NKT cells engineered for cell therapy for mammals, in particular embodiments. In such cases where the cells are NK cells, the NK cell therapy may be of any kind and the NK cells may be of any kind. In specific embodiments, the cells are NK cells that have been engineered to express one or more CARs and/or one or more suicide genes and/or one or more cytokines. In specific embodiments, the cells are NK cells that are transduced with a CAR.

In particular embodiments, the present disclosure contemplates, in part, CAR-expressing cells, CAR constructs, CAR nucleic acid molecules and CAR vectors that can be administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in NK cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a CAR, a nucleic acid sequence, a vector, as contemplated herein and/or produced by a process as contemplated herein.

Possible indications for administration of the composition(s) of the exemplary CAR cells are cancerous diseases, including tumorous diseases, including B cell malignancies, multiple myeloma, breast cancer, glioblastoma, renal cancer, pancreatic cancer, or lung cancer, for example. Exemplary indications for administration of the composition(s) of antigen-targeting CAR cells are cancerous diseases, including any malignancies that express the antigen. The administration of the composition(s) of the disclosure is useful for all stages (I, II, III, or IV) and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a CAR construct as defined herein, or components encompassed herein, a nucleic acid sequence as defined herein, a vector as defined herein and/or a host cell (such as an immune cell) as defined herein. In specific embodiments, the kit comprises nucleic acids that encode (a) CD28 hinge, (b1) CD28 transmembrane domain or (b2) DAP10 transmembrane domain, and/or (c) DAP10 costimulatory domain, or suitable primers to amplify same. It is also contemplated that the kit of this disclosure comprises a pharmaceutical composition as described herein above, either alone or in combination with further medicaments to be administered to an individual in need of medical treatment or intervention.

### VII. Pharmaceutical Compositions

Pharmaceutical compositions of the present disclosure comprise an effective amount of compositions comprising NK cells dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises the compositions will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The compositions comprising the NK cells may be formulated into a composition in a free base, neutral or salt form. Pharmaceutically acceptable salts, where appropriate include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include compositions comprising the NK cells and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the compositions comprising the NK cells and antibodies may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The therapeutic compositions comprising the NK cells of the disclosure may be administered by infusion, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In particular embodiments, the dose for delivery to an individual in need thereof, including at least by infusion, is 105 to 1010 cells/kg/dose/week, and any range derivable therein.

The quantity to be administered, both according to number of treatments and unit dose, depends on the treatment effect desired. An effective dose is understood to refer to an amount necessary to achieve a particular effect. In the practice, in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

### A. Alimentary Compositions and Formulations

In particular embodiments of the present disclosure, the compositions comprising the NK cells and antibodies are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other particular embodiments of the disclosure, the active compound compositions comprising the NK cells and antibodies may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VIII. Combination Therapies

In certain embodiments, the compositions and methods of the present embodiments involve an immune cell population (including NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

In particular embodiments, in addition to the inventive cell therapy of the disclosure, the individual may have been provided, may be provided, and/or may will be provided a specific additional therapy for cancer, including one or more of surgery, radiation, immunotherapy (other than the cell therapy of the present disclosure), hormone therapy, gene therapy, chemotherapy, and so forth.

An immune cell therapy may be administered before, during, after, or in various combinations relative to an additional cancer therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IX. Kits of the Disclosure

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit, and they may or may not yet express a CAR comprising (a) CD28 hinge, (b1) CD28 transmembrane domain or (b2) DAP10 transmembrane domain, and (c) DAP10 costimulatory domain, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more CARs, suicide gene products, and/or cytokines may be included in the kit. Proteins, such as cytokines or antibodies, including monoclonal antibodies, may be included in the kit. Nucleotides that encode components or all of engineered CAR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### X. EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1

### Car Construct Components Having Improved Signaling

FIGS. 1A-1B demonstrate that the DAP10 signaling domain confers an activated phenotype to CD5 CAR-NK cells, and phenotyping was shown using a mass cytometry panel. Using t-distributed stochastic neighbor embedding (TSNE), a statistical method for visualizing high-dimensional data by giving each datapoint a location in a two or three-dimensional map, different clusters are produced compared to non-transduced NT cells (left) and NK cells expressing a CD5 CAR comprising the IgG hinge, CD28 transmembrane domain, DAP10, and CD3zeta (CD5CAR-NK CD28TMDAP10CD3z) (FIG. 1A). Clusters 8 and 11, the 2 new clusters expressed in CARCD5 NK cells, are highlighted by the circles and rectangles. The heatmap in FIG. 1B indicates normalized expression of various markers (indicated on the X axis) in each cluster (indicated on the Y axis). The activation, cytotoxicity and maturation markers with high expression in clusters #8 and #11 are highlighted with the blue rectangles. This shows that the DAP10-transduced NK cells have two populations or clusters that are not present in non-transduced NK cells. These closers contain NK cells that express higher markers of activation, such as granzyme B, perforin, etc.

FIGS. 2A-2C demonstrate that CD5 CAR-NK cells with the DAP10 costimulatory domain are capable of producing multiple effector cytokines and chemokines and show enhanced polyfunctionality. FIG. 2A demonstrates isoplexis single cell secretome data showing polyfunctionality of CD5 CAR-NK cells with DAP10 costimulatory domain comparing different CD5 CAR-NK cells with non-transduced (NT) NK cells. CD5 CAR #5 (comprising the IgG hinge, CD28 transmembrane domain, DAP10, and CD3z) shows the highest polyfunctionality with the highest percentage of single cells secreting 2, 3, 4 or 5+ proteins at a time. FIG. 2B provides a bar graph showing the polyfunctionality strength index among the different CD5 CAR-NK cells compared to non-transduced (NT) NK cells. Here, CD5 CAR #5 also demonstrates the highest polyfunctionality with the highest proportion of effector and chemoattractive cytokines. A polyfunctionality heatmap illustrates that CD5 CAR-NK cells with the DAP10 costimulatory domain have the highest ability to secrete various permutations of cytokines at the single cell level (FIG. 2C).

CD5 CAR-NK cells with both DAP10 TM and DAP10 costimulatory domain continue to kill CD5+T-ALL cell lines (CCRF) after multiple rechallenges in an Incucyte killing assay (FIGS. 3A-3C). One schematic of a Incuycte killing assay rechallenge study is provided in FIG. 3A. The measure of the red count (a measure of live tumor count) after each tumor rechallenge (indicated by the pink arrow) among the various CD5 CAR-NK cell conditions is provided in FIG. 3B. The percent confluence, a measure of tumor abundance, following each tumor rechallenge (timing indicated by the pink arrows) is provided in FIG. 3C. These data show that CD5 CAR-NK cells with DAP10 TM and DAP10 costimulatory domain (represented by the red lines with squares) continue to kill CD5+T-ALL cell line (CCRF) after multiple rechallenges compared to other CD5 CAR NK cell designs and compared to NT NK cells and compared to irrelevant CD19 CAR-NK cells.

CD5 CAR-NK cells with DAP10 TM and DAP10 costimulatory domain exhibit higher metabolic fitness compared the other construct designs (FIGS. 4A-4C), based on a seahorse metabolic assay measuring oxygen consumption rate (OCR) and extracellular acidification rate (ECAR) among various CD5 CAR-NK cells. In FIG. 4A, OCR among the various CD5 CAR-NK cell designs is compared to non-transduced (NT) NK cells. The ECAR among the various CD5 CAR-NK cell designs is also compared to non-transduced (NT) NK cells (FIG. 4B). These data demonstrate significantly higher OCR for CD5 CAR-NK cells with DAP10 TM and DAP10 costimulatory domains compared to other designs and compared to NT NK cells, in addition to the same construct having among the highest ECAR that imparts them with a higher metabolic fitness.

FIGS. 5A-5C shows that CD5 CAR-NK cells with DAP10 costimulatory domain improve tumor control in a PDX mouse model of CD5+ Mantle cell lymphoma. The absolute number is shown of CD45+CD5+ cells (in the subcutaneous tumor in mice who received tumor alone (on the left) vs tumor plus CD5CAR-NK (on the right) in the subcutaneous tumor (FIG. 5A). FIG. 5B provides a bar graph showing the absolute number of CD45+CD5+ cells in the subcutaneous tumor in mice who received tumor alone (left) vs tumor plus CD5CAR-NK (right) in the spleen. The absolute number is shown of CD45+CD5+ cells in the subcutaneous tumor in mice who received tumor alone (left) vs tumor plus CD5CAR-NK (right) in the bone marrow (FIG. 5C).

FIGS. 6A-6B demonstrate that CD27 CAR-NK cells with DAP10 costimulatory domain improve tumor control and survival is an NSG mouse model of acute myeloid leukemia (THP-1 transduced with firefly luciferase (FFLuc)). In FIG. 6A, a series of bioluiminescent imaging (BLI) shows tumor burden as luminescence of THP-1 FFLuc among the various groups of mice. A survival curve showing the survival of the various groups of mice over time is provided in FIG. 6B indicating that the DAP10 costimulatory domain improves the in vivo potency of CD27 CAR-NK cells.

### Example 2

### Superiority of the CD28Hinge-CD28 Transmembrane Domain-DAP10 Costimulatory Domain Car

The present example concerns particular CAR constructs for characterization, including various constructs that comprise the CD28 hinge, some that comprise the CD28 transmembrane domain, and some that comprise DAP10 costimulatory domain. In one particular embodiment, activity is provided for a CAR construct comprising the CD28 hinge, the CD28 transmembrane domain, and the DAP10 costimulatory domain.

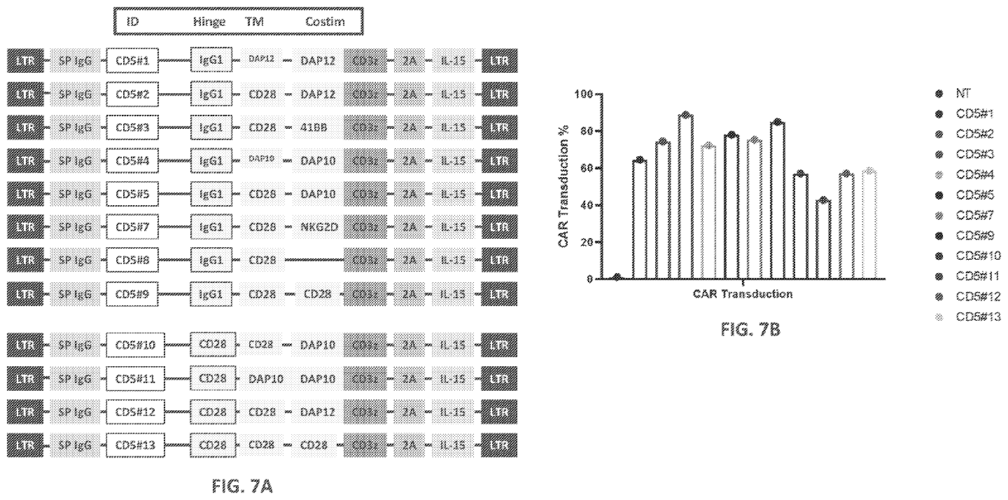

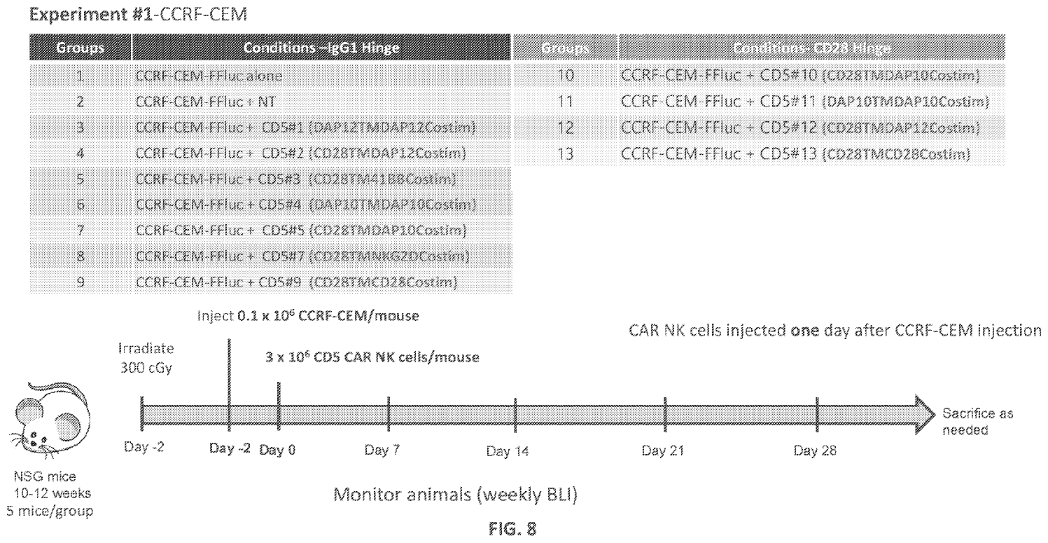

FIG. 7A illustrates various construct identifications and corresponding transduction efficiency (FIG. 7B). CB-NK cells were transduced with various CD5 CAR constructs, as shown in FIG. 7A, and the transduction efficiency was measured by flow cytometry. The transduction efficiency is based on percent positive cells (FIG. 7B).

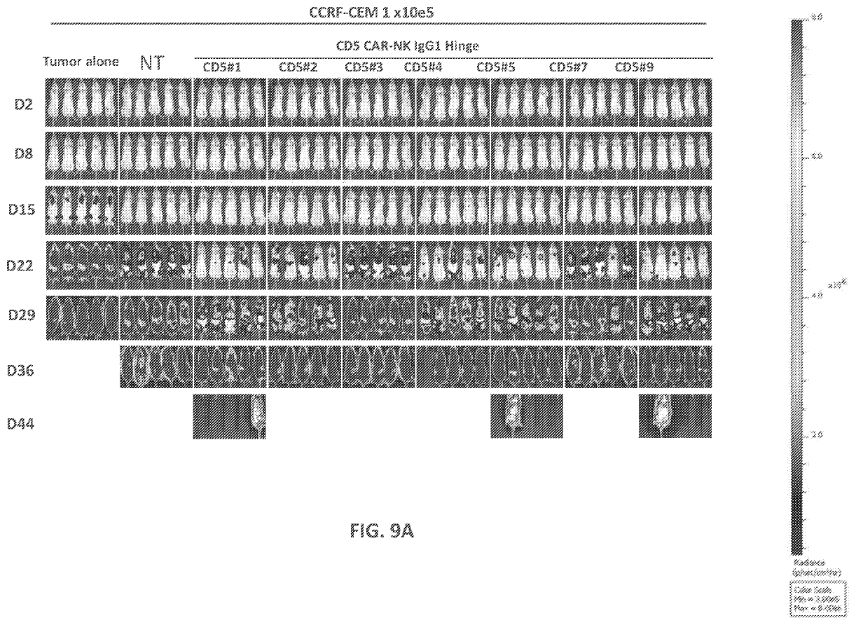

FIG. 8 provides one example of an experimental plan for mice injection with various CD5 constructs and a corresponding timeline. The schematic shows testing of the in vivo antitumor activity of various CD5 CAR NK cells against the T lymphoblastoid cell line CCRF-CEM as a target.

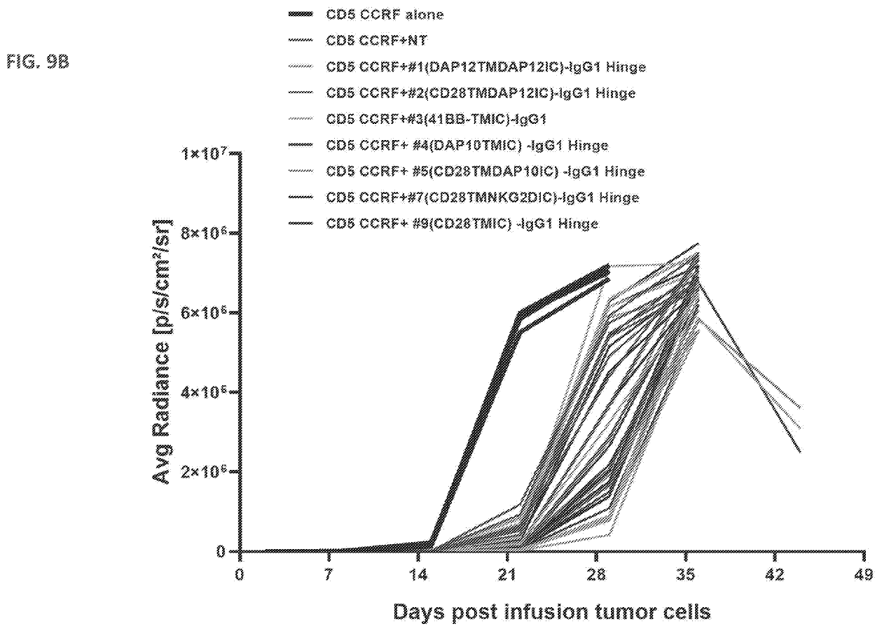

FIGS. 9A and 9B show that mice treated with anti-CD5 CAR NK with IgG1 hinge cells survive significantly longer than NT NK cell and Tumor alone. CD5 CAR NK Cells reduced the tumor burden in a mouse model of T-acute lymphocytic leukemia. CCRF-CEM cells, transduced with firefly luciferase (FFLuc) were injected into mice with 1×10⁵/mouse, and were monitored with bioluminescence imaging among the various groups. Mice in treatment group were injected with 3M of respective NK CAR cells, 2 days post tumor injection. Bioluminescence images (FIG. 9A) of mice in each groups, and quantification (FIG. 9B) of luciferase signal shows that mice receiving various CD5 NK CAR with IgG1 hinge displayed enhanced CCRF-CEM tumor control when compared to tumor alone, NT NK cells but they succumb to tumor by time because of lack of persistence of NK cells.

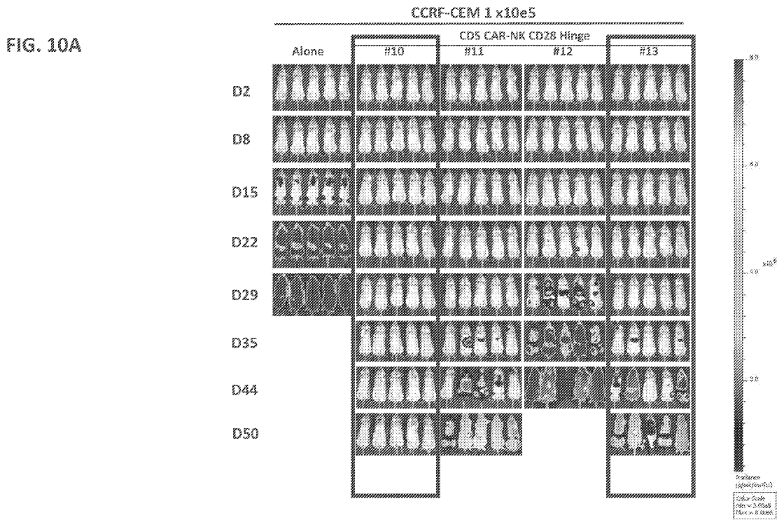

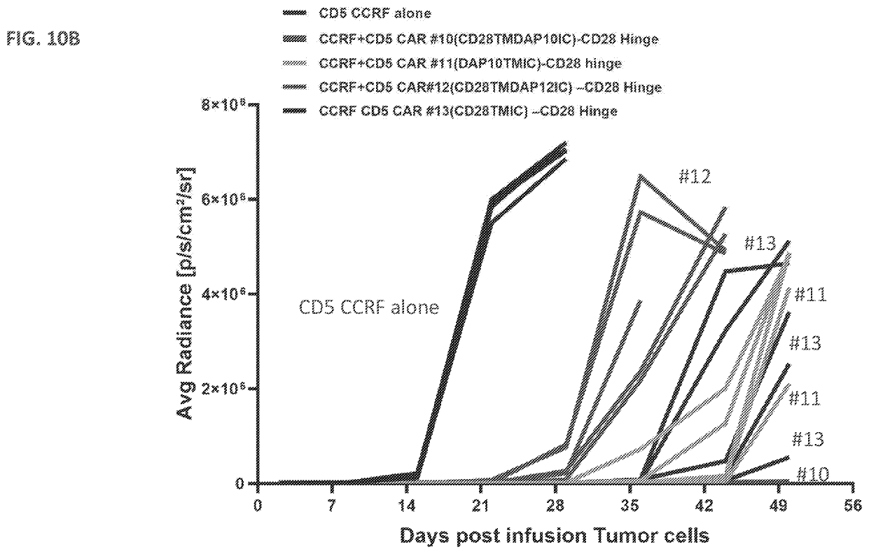

In FIGS. 10A and 10B demonstrate that mice treated with anti-CD5 CAR NK with CD28 hinge reduce tumor burden significantly compared to Tumor alone, NT NK cells and CD5 CAR NK cells with IgG1 hinge. CD5 CAR NK Cells reduced the tumor burden in a mouse model of T-acute lymphocytic leukemia. CCRF-CEM cells, transduced with firefly luciferase (FFLuc) were injected into mice with 1×10⁵/mouse and were monitored with bioluminescence imaging among the various groups. Mice in treatment group were injected with 3M of respective NK CAR cells, 2 days post tumor injection. Bioluminescence images (FIG. 10A) of mice in each groups, and quantification (FIG. 10B) of luciferase signal shows that mice receiving various CD5 NK CAR with CD28 hinge displayed enhanced CCRF-CEM tumor control when compared to tumor alone, NT NK cells and CD5 NK CAR with IgG1 hinge. Further, DAP10 costimulatory domain CD5 NK CAR improve tumor control significantly when compared with other costimulatory domains.

### Example 3

CD5 Car-NK Cells with DAP10 Signaling Show a Signature of High Proliferation Capacity, Metabolic Activity and Memory Features at the Transcriptomic, Epigenetic and Proteomic Levels

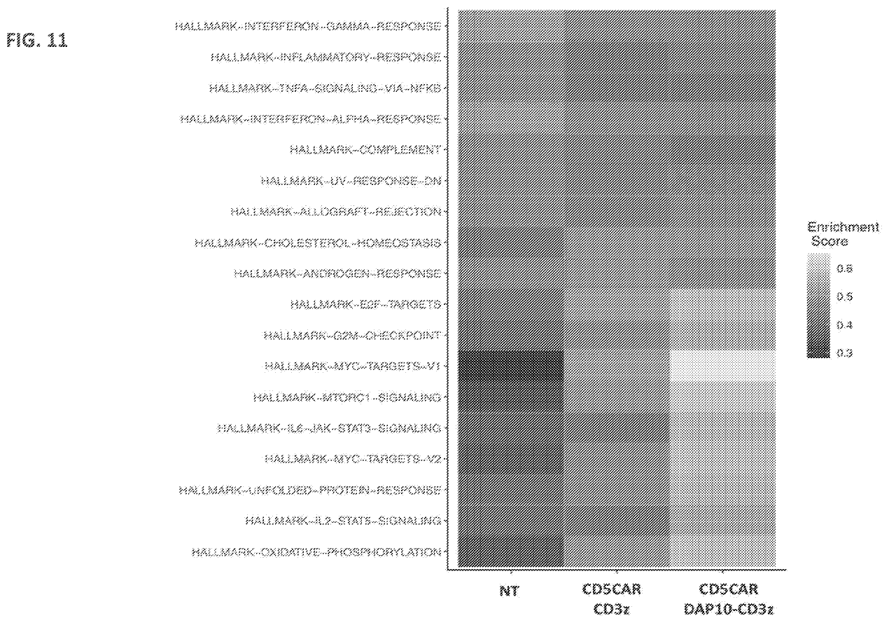

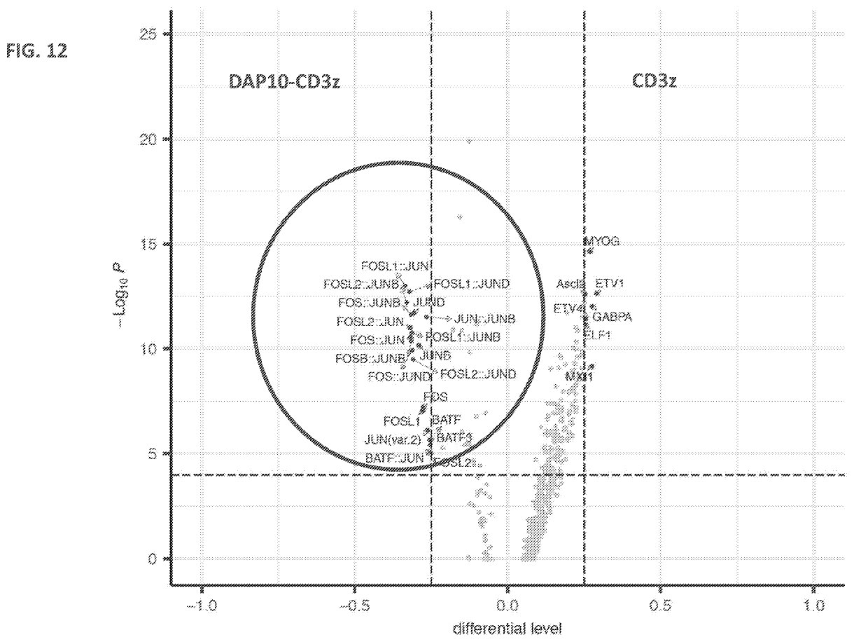

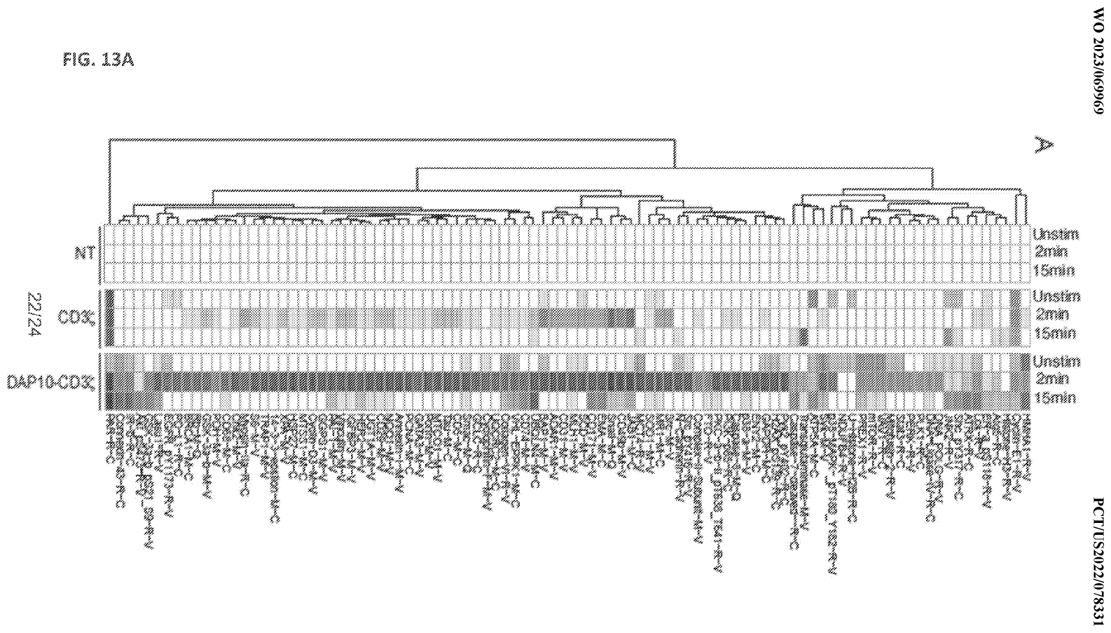

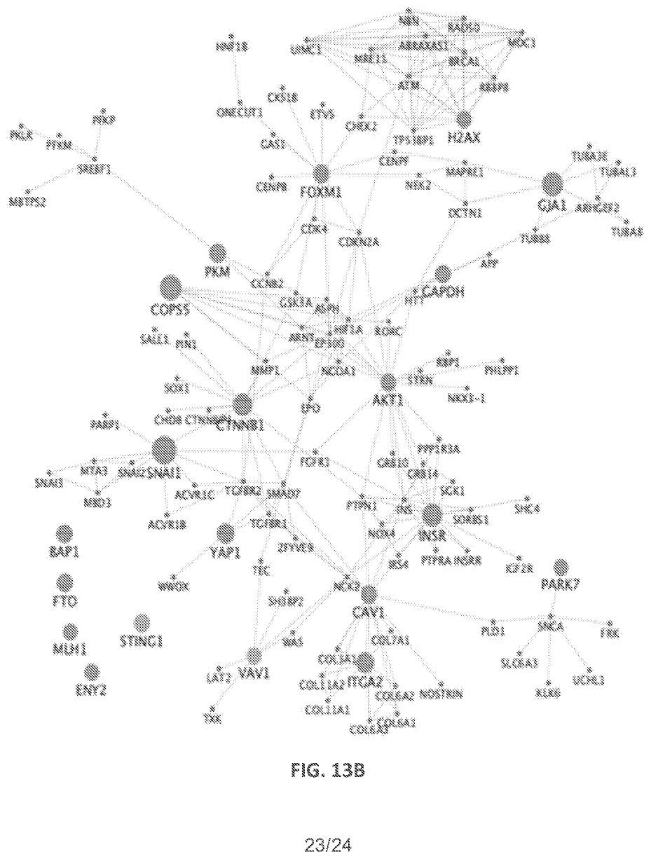

The CD5 CAR construct that includes CD28 transmembrane (TM) domain and DAP10 co-stimulatory domain with CD3z signaling domain (CD28TMDAP10CD3ζ) performs better than other examples of CD5 CAR-NK cell constructs incorporating other costimulatory molecules, showing superior in vitro and in vivo anti-tumor activity, enhanced polyfunctionality and metabolic fitness, and less functional exhaustion following multiple tumor rechallenges in vitro. To further characterize why DAP10 signaling is providing this significant advantage to the CAR-NK cells, the inventors performed single cell RNA sequencing (scRNAseq), single cell ATAC sequencing (scATACseq) and reverse phase protein arrays (RPPA) comparing CD5 CAR-NK cells with DAP10-CD3ζ signaling to CD5 CAR-NK cells designed without a costimulatory domain (CD3ζ signaling only) and to non-transduced (NT) NK cells generated from the same donor. At the single cell transcriptomic level, pathway enrichment analysis of scRNAseq data showed that DAP10 signaling endows CAR-NK cells with superior proliferative capacity as evidenced by enrichment in E2F targets and G2M checkpoint pathways, as well as IL-2/STAT5 signaling and enhanced metabolic activity as evidenced by enrichment of metabolic pathways such as Myc, mTORC1 and oxidative phosphorylation (FIG. 11). On the epigenetic level, the scATACseq data show that CD5 CAR-NK cells with DAP10-CD3z signaling domain show enrichment in AP-1 complex and BATF transcription factors related to memory formation and exhaustion resistance respectively (FIG. 12). On the proteomic level, pathway enrichment analysis of RPPA data corroborated the transcriptomic results showing that following stimulation with the CD5 target antigen DAP10 signaling boosts the CAR-NK cells ability to proliferate and produce cytokines (ITGA and INSR protein pathways), provides them with stemness potential (COPS5 pathway), enhances their metabolic activity at both the glycolytic and mitochondrial levels (GAPDH and PARK7 pathways), augments their membrane polarization and capacity to form immune synapses with target cells (Cav1 pathway), and endows them with memory potential (FOXM1 pathway) (FIGS. 13A and 13B).

### Example 4

CD5 Car-NK Cells with DAP10 Signaling Persist and Show Evidence of Memory Response In Vivo Following Tumor Rechallenge.

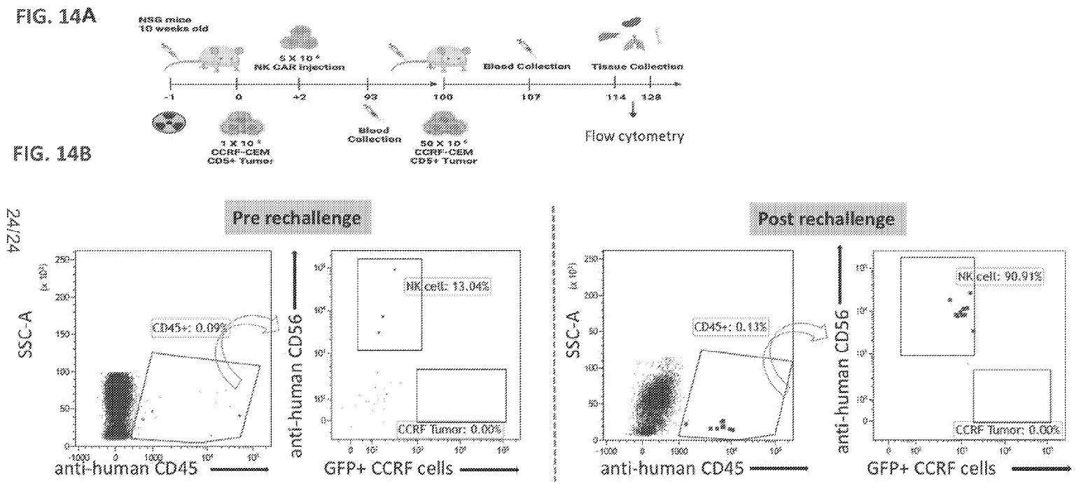

The CAR-NK cells were evaluated for the ability to mount a memory response in vivo following tumor rechallenge. A well-established NSG mouse model was used of CCRF-CEM, a CD5+T-ALL cell line. The CCRF-CEM tumor cell line was transduced with fireflyluciferase and GFP (CCRF-Ffluc-GFP) to be able to monitor the tumor growth by bioluminescent imaging (BLI) and by flow respectively. Mice were irradiated (225cGy) on day −1 and then injected intravenously with CCRF-Ffluc tumor 100,000 cells per mouse on day 0. On day 2, the treatment group received an intravenous injection of 5×10⁶ CD5 CAR-NK cells with DAP10 signaling. On day 93 following CCRF-CEM tumor injection, blood was collected to evaluate the percentage of human NK cells (hCD45+CD56+GFP−). On day 100, mice that were cured and did not show evidence of tumor by BLI or by flow were rechallenged with 50,000 CCRF-CEM tumor cells. 1 week later (on day 107), blood was collected again to check for human NK cell percentage. As seen in FIGS. 14A and 14B, following tumor rechallenge, the percentage of human NK cells (hCD45+CD56+GFP−) in the blood increases from 13% to 91% indicating that the CAR-NK cells are able to mount a memory response to tumor rechallenge in vivo.

### REFERENCES

The following references, to the extent that they provide exemplary procedural or other details supplementary to those set forth herein, are specifically incorporated herein by reference.
1. Maude S L, Laetsch T W, Buechner J, et al. Tisagenlecleucel in Children and Young Adults with B-Cell Lymphoblastic Leukemia. N Engl J Med. 2018; 378(5):439-448.
2. Neelapu S S, Locke F L, Bartlett N L, et al. Axicabtagene Ciloleucel CAR T-Cell Therapy in Refractory Large B-Cell Lymphoma. N Engl J Med. 2017; 377(26):2531-2544.
3. Schuster S J, Bishop M R, Tam C S, et al. Tisagenlecleucel in Adult Relapsed or Refractory Diffuse Large B-Cell Lymphoma. N Engl J Med. 2019; 380(1):45-56.
4. Hartmann J, Schussler-Lenz M, Bondanza A, Buchholz C J. Clinical development of CAR T cells-challenges and opportunities in translating innovative treatment concepts. EMBO Mol Med. 2017; 9(9):1183-1197.
5. Daher M, Rezvani K. Next generation natural killer cells for cancer immunotherapy: the promise of genetic engineering. Curr Opin Immunol. 2018; 51:146-153.
6. Mehta R S, Rezvani K. Chimeric Antigen Receptor Expressing Natural Killer Cells for the Immunotherapy of Cancer. Front Immunol. 2018; 9:283.
7. Liu E A S, Kerbauy L et al. GMP-compliant universal antigen presenting cells (uAPC) promote the metabolic fitness and antitumor activity of armored cord blood CAR-NK cell. Front Immunol doi: 103389/fimmu2021626098. 2021.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polynucleotide that encodes a fusion protein, said fusion protein comprising:
(a) optionally, a hinge; and
(b1) a CD28 transmembrane domain, or
(b2) a DAP10 transmembrane domain;
(c) a DAP10 costimulatory domain; and
(d) CD3zeta.

2. The polynucleotide of claim 1, wherein the fusion protein is further defined as a chimeric antigen receptor (CAR).

3. The polynucleotide of claim 2, wherein when an extracellular domain of the CAR comprises an scFv, the CAR comprises a hinge.

4. The polynucleotide of claim 2, wherein when an extracellular domain of the CAR comprises part or all of an extracellular domain of a receptor, the CAR lacks a hinge.

5. The polynucleotide of claim 2, wherein when an extracellular domain of the CAR comprises part or all of an extracellular domain of a receptor, the CAR comprises a hinge.

6. The polynucleotide of claim 2, wherein the CAR further comprises one or more antigen binding domains.

7. The polynucleotide of claim 6, wherein an antigen binding domain targets a tumor antigen or an infectious agent.

8. The polynucleotide of any one of claims 1-7, wherein the CD3zeta comprises SEQ ID NO:3.

9. The polynucleotide of any one of claims 1-8, wherein the CD28 transmembrane domain comprises SEQ ID NO:1.

10. The polynucleotide of any one of claims 2-9, wherein the CAR further comprises one or more additional costimulatory domains.

11. The polynucleotide of claim 10, wherein the one or more additional costimulatory domains are selected from the group consisting of CD28, DAP12, 4-1BB, NKG2D, 2B4, and a combination thereof.

12. The polynucleotide of any one of claims 2-11, wherein the CAR further comprises a signal peptide.

13. The polynucleotide of claim 12, wherein the signal peptide is a signal peptide from CD8, CD27, granulocyte-macrophage colony-stimulating factor receptor (GMSCF-R), Ig heavy chain (IgH), CD3, or CD4.

14. The polynucleotide of any of claims 2-13, wherein the polynucleotide further encodes an additional polypeptide of interest.

15. The polynucleotide of claim 14, wherein the sequence encoding the additional polypeptide of interest and the sequence encoding the CAR are separated on the polynucleotide by a 2A element.

16. The polynucleotide of claims 14 or 15, wherein the additional polypeptide of interest is a therapeutic protein or a protein that enhances cell activity, expansion, and/or persistence.

17. The polynucleotide of any one of claims 14-16, wherein the additional polypeptide of interest is a suicide gene product, one or more cytokines, or one or more human or viral proteins that enhance proliferation, expansion and/or metabolic fitness.

18. The polynucleotide of claim 17, wherein the cytokine is IL-15, IL-2, IL-12, IL-18, IL-21, IL-23, or IL-7.

19. The polynucleotide of claim 17 or 18, wherein the cytokine is IL-15.

20. The polynucleotide of claim 18 or 19, wherein the IL-15 sequence comprises SEQ ID NO:8.

21. A vector comprising the polynucleotide of any one of claims 1-20.

22. The vector of claim 21, wherein the vector is a viral vector.

23. The vector of claim 22, wherein the viral vector is an adenoviral vector, adeno-associated viral vector, lentiviral vector, or retroviral vector.

24. The vector of claim 21, wherein the vector is a non-viral vector.

25. The vector of claim 24, wherein the non-viral vector is a plasmid.

26. A cell comprising the polynucleotide of any one of claims 1-20 or the vector of any one of claims 21-25.

27. The cell of claim 26, wherein the cell is an immune cell.

28. The immune cell of claim 27, wherein the immune cell is a natural killer (NK) cell, T cell, gamma delta T cell, alpha beta T cell, invariant NKT (iNKT) cell, B cell, macrophage, mesenchymal stromal cell, or dendritic cell.

29. The immune cell of claim 27, wherein the immune cell is a NK cell.

30. The immune cell of claim 29, wherein the NK cell is derived from cord blood, peripheral blood, induced pluripotent stem cells, hematopoietic stem cells, bone marrow, or from a cell line.

31. The immune cell of claim 30, wherein the NK cell is derived from a cell line, wherein the NK cell line is NK-92.

32. The immune cell of claim 30, wherein the NK cell is derived from a cord blood mononuclear cell.

33. The immune cell of any one of claims 28-32, wherein the NK cell is a CD56+NK cell.

34. The immune cell of any one of claims 28-33, wherein the NK cell expresses a recombinant cytokine.

35. The immune cell of claim 34, wherein the cytokine is IL-15, IL-2, IL-12, IL-18, IL-21, IL-7, or IL-23.

36. A population of immune cells comprising the immune cell of any one of claims 27-35.

37. A method of killing cancer cells in an individual, comprising administering to the individual an effective amount of cells harboring the polynucleotide of any one of claims 1-20 or cells harboring the vector of any one of claims 21-25.

38. The method of claim 37, wherein the cells harboring the polynucleotide are immune cells.

39. The method of claim 38, wherein the immune cells are NK cells, T cells, gamma delta T cells, alpha beta T cells, iNKT cells, B cells, macrophages, dendritic cells, or a mixture thereof.

40. The method of claim 38 or 39, wherein the immune cells comprise NK cells, wherein the NK cells are derived from cord blood, peripheral blood, induced pluripotent stem cells, hematopoietic stem cells, bone marrow, from a cell line, or a mixture thereof.

41. The method of claim 39 or 40, wherein the NK cells are derived from cord blood mononuclear cells.

42. The method of any one of claims 38-41, wherein the immune cells are allogeneic with respect to the individual.

43. The method of any one of claims 38-41, wherein the immune cells are autologous with respect to the individual.

44. The method of any one of claims 37-43, wherein the cells harboring the polynucleotide or cells harboring the vector are administered to the individual once or more than once.

45. The method of claim 44, wherein the duration of time between administrations of the cells harboring the polynucleotide to the individual is 1-24 hours, 1-7 days, 1-4 weeks, 1-12 months, or one or more years.

46. The method of any one of claims 37-45, further comprising the step of providing to the individual an effective amount of an additional therapy.

47. The method of claim 46, wherein the additional therapy comprises surgery, radiation, gene therapy, immunotherapy, or hormone therapy.

48. The method of any one of claims 37-47, wherein the cells harboring the polynucleotide or the cells harboring the vector are administered to the individual by infusion, injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.


SEQUENCE LISTING (14 sequences)

SEQ ID NO: 1 - protein, 27 aa, synthetic construct
FWVLVVVGGVLACYSLLVTVAFIIFWV

SEQ ID NO: 2 - protein, 24 aa, synthetic construct
LCARPRRSPAQEDGKVYINMPGRG

SEQ ID NO: 3 - protein, 113 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 4 - protein, 42 aa, synthetic construct
KRGRKKLLYIFKQPFMRPVQTTQEEDGCSCRFPEEEEGGCEL

SEQ ID NO: 5 - protein, 52 aa, synthetic construct
YFLGRLVPRGRGAAEAATRKQRITETESPYQELQGQRSDVYSDLNTQRPYYK

SEQ ID NO: 6 - protein, 51 aa, synthetic construct
SANERCKSKVVPCRQKQWRTSFDSKKLDLNYNHFESMEWSHRSRRGRIWGM

SEQ ID NO: 7 - protein, 39 aa, synthetic construct
IEVMYPPPYLDNEKSNGTIIHVKGKHLCPSPLFPGPSKP

SEQ ID NO: 8 - protein, 160 aa, synthetic construct
ISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 402 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 10 - protein, 20 aa, synthetic construct
QCTNYALLKLAGDVESNPGP

SEQ ID NO: 11 - protein, 18 aa, synthetic construct
EGRGSLLTCGDVEENPGP

SEQ ID NO: 12 - protein, 19 aa, synthetic construct
ATNFSLLKQAGDVEENPGP

SEQ ID NO: 13 - protein, 22 aa, synthetic construct
VKQTLNFDLLKLAGDVESNPGP

SEQ ID NO: 14 - protein, 41 aa, synthetic construct
RSKRSRLLHSDYMNMTPRRPGPTRKHYQPYAPPRDFAAYRS